(19) United States
(12) Patent Application Publication
(10) Pub. No.: US 2004/0147595 A1
(43) Pub. Date: Jul. 29, 2004

(54) Biofilm degrading or sloughing compositions and methods

(76) Inventors: Steffan Kjelleberg, La Perouse NSW (AU); Michael Givskov, Humlebaek (DK); Mortan Hentzer, Frederlisberg (DK)

Correspondence Address: NIXON & VANDERHYE, PC, 1100 N GLEBE ROAD, 8TH FLOOR, ARLINGTON, VA, 22201-4714, US

(21) Appl. No.: 10/481,250

(22) PCT Filed: Jun. 18, 2002
(86) PCT No.: PCT/AU02/00797

(30) Foreign Application Priority Data
Jun. 18, 2001 (AU) PR5754

(51) Int. Cl. A61K 31/365

(52) U.S. Cl. 514/463

(57) ABSTRACT

The present invention relates to a method for the regulation and control of biofilm layers. In particular, the present invention is concerned with methods for degrading or causing sloughing of biofilms from surfaces. The invention is also related to compositions suitable for use in carrying out these methods.

### FIELD OF THE INVENTION

The present invention relates to a method for the regulation and control of biofilm layers. In particular, the present invention is concerned with methods for degrading or causing sloughing of biofilms from surfaces. The invention is also related to compositions suitable for use in carrying out these methods.

### BACKGROUND OF THE INVENTION

Biofilms are biological films that can develop and persist on solid substrates in contact with moisture, on soft tissue surfaces in living organisms and at liquid air interfaces. They can develop into structures several millimetres or centimeters in thickness and can cover large surface areas. They may contain either single or multiple microbial species and readily adhere to such diverse surfaces as river rocks, soil pipelines, teeth, mucous membranes, and medical implants.

Biofilms form along inner walls of ping conduits in industrial facilities and in household plumbing systems. They can play a role in restricting or entirely blocking the flow in the plumbing systems and can decrease the life of materials through corrosive action mediated by embedded bacteria. Biofilms can also result in the reduction of the efficiency of industrial processes, wasting energy, and reducing product quality.

Biofilms frequently cause problems in cooling water systems used in power-generating plants, refineries, chemical plants, and air conditioning systems. Cooling water systems are often contaminated with airborne organisms entrained by air/water contact in cooling towers as well as waterborne organisms from the system's makeup water supply. Biofilms can also comprise water supplies in that they can provide a haven far disease causing microorganisms that can proliferate despite chlorination.

The control and removal of biofilm material from piping conduit surface has historically been carried out by the addition of corrosive chemicals such as chlorine or strong alkalis or through mechanical means. Such treatments are generally harsh to both the equipment and the environment and have been necessary due to the recalcitrant nature of biofilms within those systems. The resistance to treatment has been due in large measure to the protective character of intact biofilm matrix polymers.

Biofilm formation a has implications in human and animal health Biofilms can present a serious threat to health as foci of chronic infections. For example, biofilm composed of Pseudomonas aeruginosa, the bacterium responsible for biofilm formed in the lungs of cystic fibrosis patients, is believed to be behind the fatal lung infections in patent suffering this disease. Biofilms have been implicated in periodontal disease, tooth decoy, prostate infections, kidney stones, tuberculosis. Legionnaire's disease and some infections of the middle ear.

Biofilms may also be the cause of infections resulting from medical intervention. For example, biofilms can form on medical devices including catheters, medical implants, dental equipment and contact lenses.

Commonly, patients with indwelling catheters for urine excretion, for continuous ambulatory peritoneal dialysis (CAPD) or for any other reason are subject to frequent and persistent bouts of infection. These recurrent infections are due to the accumulation of mixed biofilms on the artificial surfaces provided by the catheter or other implant. The glycocalyx in which the bacteria live protects them from the effects of antibiotics and accounts for the persistence of the infection even in the face of vigorous chemotherapy.

Biofilm formation can be a serious complication in bioimplants such as bone prosthesis, heart valves, pacemakers, stents, orthopaedic devices, ear implant devices, electrodes, dialysis devices and the like. Biofilm formation on exposed surfaces of a bioimplant can degrade the function of the implant, as in the case of implanted valves, load to serious joint or bone infections, as in the case of a bone prosthesis, and in all cases, provide a source of difficult to treat septic infection.

Infections due to microbial keratitis, ecanthamoeba or ulcerative keratitis are recurring problems associated with contact lens wear. The problems may arise for example when a contact lens is not cleansed sufficiently by the lens wearer, and the bacterial load of the lens increases such that a biofilm forms on the lens. In such cases not all lens cleaning solutions may be strong enough to kill residual bacteria. Similarly the contact lens may harbour infectious organisms such as acanthamoeba, which can also contaminate the lens case in addition to the lens resulting in time in a devastating keratitis.

Biofilm-derived dental unit waterline contamination is a problem in the dental industry. The formation at biofilms provides the potential for exposure of dental personnel and patients to high concentrations of microbes that may present a risk of infection.

Prevention of colorization by and eradication of biofilm-associated microorganisms is an important, and often difficult to solve problem in medicine. The extracellular materials (polysaccharide, proteins, etc) that make up the biofilm can be a problem in itself, eg, blockage of a catheter or by causing a spurious immune response. Generally though, the problem is that the cells within the biofilm are more resistant to a number of treatments. For example, P. aeruginosa is 50,000 times more resistant to the drug tobramycin when in the biofilm form compared to the planktonic cells. Thus, traditional biocides are less/not effective against the biofilm population. They are also less vulnerable to the immune system and the matrix polysaccharides of the biofilms resist enzyme attack.

There is a need in the medical, environmental and industrial arts for the control of biofilm formation. The control of biofilms can be carried out more effectively if the production and regulation of exopolysaccharide material produced by the bacteria can be influenced externally.

### SUMMARY OF THE INVENTION

The present inventors have determined that furanones and related compounds can cause degradation or sloughing of biofilms.

Accordingly, in a first aspect the present invention consists in a method of degrading or causing sloughing of biofilms, the method comprising applying to the biofilm a composition comprising at least one compound of general formula I:

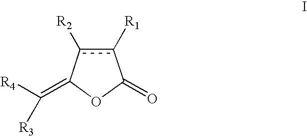

wherein R₁ is selected from H, halogen, alkyl, alkoxy, acyl, alkenyl, aryl, alkylaryl or arylalkyl whether unsubstituted or substituted, optionally interrupted by one or more heteroatoms, straight chain or branched chain, hydrophilic or fluorophilic;

R₂, R₃ and R₄, which may be the same or different, are independently selected from H, halogen, alkyl, alkoxy, acyl, alkenyl, aryl, alkylaryl, arylalkyl or a silyl group, whether unsubstituted or substituted, optionally interrupted by one or more heteroatoms, straight chain or branched chain, hydrophilic or fluorophilic.

R₃ or R₄+R₂ can be a saturated or an unsaturated cycloalkane; and

“═” represents a single bond or a double bond,

or a compound of general formula II

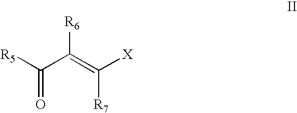

wherein R₅, R₆ and R₇, which may be the same or different, are independently selected from H, halogen, alkyl, alkoxy, alkenyl, alkynyl, aryl, arylalkyl, carboxyl, acyl, acyloxy, acylamino, formyl and cyano whether unsubstituted or substituted, optionally interrupted by one or more hetero atoms, straight chain or branched chain, hydrophilic, hydrophobic or fluorophilic and X is a halogen.

In one embodiment R₆ and R₇ are independently H, halogen, carboxyl, ester, formyl, cyano, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;

X is a halogen;

R₅ is H, alkyl, alkenyl, alkynyl, alkene, alkyne, aryl, arylalkyl, whether unsubstituted or substituted, optionally interrupted by one or more heteroatoms, straight chain or branched chain, hydrophilic or fluorophilic.

In the compound of formula I, preferably, at least one of R₁, R₂, R₃ and R₄ is bromine. Most preferably, at least one of R₃ and R₄ is Br. In the compound of formula II, preferably at least one of R₅, Ror R₇ is bromine.

The term “alkyl” is taken to mean both straight chain or branched alkyl groups such as methyl, ethyl, propyl, isopropyl, butyl, isobutyl, sec-butyl, tertiary butyl, and the like. Preferably the alkyl group is a lower alkyl of 1 to 6 carbon atoms. The alkyl group may optionally be substituted by one or more groups selected from alkyl, cycloalkyl, alkenyl, alkynyl, halo, haloalkyl, haloalkynyl, hydroxy, alkoxy, alkenyloxy, haloalkoxy, haloalkenyloxy, nitro, amino, nitroalkyl, nitroalkenyl nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocycyl, heterocycloxy, hetrocyclamino, haloheterocyclyl, alkylsulfenyl, alkylcarbonyloxy, alkylthio, acylthio, phosphorus-containing groups such as phosphono and phosphinyl. The alkyl group may also be perfluorinated.

The term “alkoxy” denotes straight chain or branched alkyloxy, preferably C₁₋₁₀ alkoxy. Examples include methoxy, ethoxy, n-propoxy, isopropoxy and the different butoxy isomers.

The term “alkenyl” denotes groups formed from straight chain, branched or mono or polycyclic alkenes and polyene. Substituents include mono- or poly-unsaturated alkyl or cycloalkyl groups as previously defined, preferably C₂₋₁₀ alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cyloheptadienyl, 1,3,5cyloheptatrienyl, or 1,3,5,7-cyclooctateraenyl.

The term “halogen” denotes fluorine, chlorine, bromine or iodine, preferably bromine or fluorine.

The term “heteroatoms” denotes O, N or S.

The term “acyl” used either alone or in compound words such as “acyloxy”, “acylthio”, “acylamino” or diacylamino” denotes an aliphatic acyl group and an acyl group containing a heterocyclic ring which is referred to as heterocyclic acyl, preferably a C1-10 alkanoyl. Examples of acyl include carbamoyl; straight chain or branched alkanoyl, such as formyl, acetyl, propanoyl, butanoyl, 2-methylpropanoyl, pentanoyl, 2,2-dimetylpropanoyl, hexanoyl, heptanoyl, octanoyl, nonanoyl, decanoyl; alkoxycarbonyl, such as methoxycarbonyl, ethoxycarbonyl, t-butoxycarbonyl, t-pentyloxycarbonyl or heptyloxycarbonyl: cycloalkanecarbonyl such as cyclopropanecarbonyl cyclobutanecarbonyl, cyclopentanecarbonyl or cyclohexanecarbonyl; alkanesulfonyl, such as methanesulfonyl or ethanesulfonyl; alkoxysulfonyl, such as methoxysulfonyl or ethoxysulfonyl; heterocycloalkanecarbonyl; heterocyclyoalkanoyl, such as pyrrolidinylacetyl, pyrrolidinylpropanoyl, pyrrolidinylbutanoyl, pyrrolidinylpentanoyl, pyrrolidnylhexanoyl or thiazolidinylacetyl: heterocyclalkenoyl, such as heterocyclylpropenoyl, heterocyclylbutenoyl, heterocyclylpentenoyl or heterocyclohexenoyl; or heterocylglyoxyoyl, such as, thiazolidinylglyoxyloyl or pyrrolidinylglyoxyloyl.

As will recognised by those skilled in the art the compounds of general formulas I and II can exist as two isomers E and Z. Furthermore, some substituents in the side chain may result in compounds of formula I or II that have optically active enantiomers. It is intended that the general formulas depicted herein are not limited to a particular isomer and encompass both isomers either in the form of a racemic mixture or separated isomers.

### DETAILED DESCRIPTION OF THE INVENTION

The biofilm to be treated may be dominated or characterised by undesirable bacterial cells, for example, living cells selected from, but not limited to, the bacterial genera Pseudomonas, Staphylococcus, Aeromonas, Burkholderia, Erwinia, Fusobacterium, Helicobacter, Klebsiella, Listeria, Mycobacterium, Neisseria, Porphyromonas, Providencia, Ralstonia, Salmonella, Staphylococcus, Streptococcus, Vibrio, Xenorhabus, and Yersinia.

The biofilm may be dominated by or characterised by, but not limited to, one or more of the organisms Aeromonas hydrophilia, A. salmonicida, Burkholderia cepacia, Enterobacter aerogenes, Escherichia coli, Erwinia carotovora, Fusobacterium nucleatum, Helicobacter pylori, Klebsiella pneumonia, Listeria monocytogenes, Mycobacterium tuberculosis, Nelsseria meningitidis, N. gonorrhea, Porphyromonas gingivalis, Providencia stuartii, Pseudomonas aeruginosa, Ralstonia solanacearum, Salmonella typhimurium, Salmonella cholerasuis, Serratia liquefaciens, S. marcesens, Staphylococcus aureus, S. epidermidis, Streptococcus mutans (sobrinus), Strep. pyogenes, Strep pneumonia, Vibrio parahaemolyticus, V. vulnificus, V. cholerae, V. harveyi, V. anguillarum, Xenorhabus nemotophilus, Yerslnia pestis, Y. enterocolitica, Y. pseudotuberculosis.

In an embodiment of the present invention the microorganism constituting the biofilm is Pseudomonas sp., particularly Pseudomonas aeruginosa.

In a further preferred embodiment the composition comprises at least one compound 30 or 56 as set out in Table 1.

Pseudomonas biofilms are of particular concern in cystic fibrosis.

Accordingly, in a second aspect the present invention consists in a method of degrading or causing sloughing of a Pseudomonas biofilm in the lung of a subject suffers from cystic fibrosis, the method comprising administering to the biofilm a composition comprising at least one compound of general formula I:

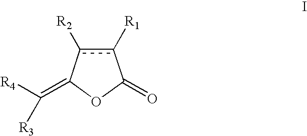

wherein R₁ is selected from H, halogen, alkyl, alkoxy, acyl, alkenyl, aryl, alkylaryl or arylalkyl whether unsubstituted or substituted, optionally interrupted by one or more heteroatoms, straight chain or branched chain, hydrophilic or fluorophilic;

R₂, R₃ and R₄, which may be the same or different, are independently selected from H, halogen, alkyl, alkoxy, acyl, alkenyl, aryl, alkylaryl, arylalkyl, or a silyl group, whether unsubstituted or substituted optionally interrupted by one or more heteroatoms, straight chain or branched chain, hydrophilic or fluorophilic,

R₃ or R₄+R₂ can be a saturated or an unsaturated cycloalkane; and

“═” represents a single bond or a double bond.

or a compound of general formula II

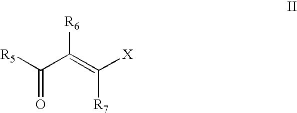

wherein R₆ and R₇ are independently H, halogen, carboxyl, ester, formyl, cyano, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;

X is a halogen;

R₅ is H, alkyl, alkenyl, alkynyl, alkene, alkyne, aryl, arylalkyl, whether unsubstituted or substituted, optionally interrupted by one or more heteroatoms, straight chain or branched chain, hydrophilic or fluorophilic.

As used herein the terms “degrading” or “sloughing” are intended to convey that the thickness of the biofilm is reduced or that the biofilm is disrupted.

In a third aspect, the present invention provides a biofilm degrading or sloughing composition comprising an amount of a compound comprising at least one compound of formula I:

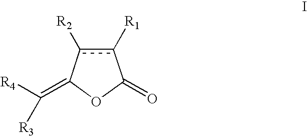

wherein R₁ is selected from H, halogen, alkyl, alkoxy, acyl, alkenyl, aryl, alkylaryl or arylalkyl whether unsubstituted or substituted, optionally interrupted by one or more heteroatoms, straight chain or branched chain, hydrophilic or fluorophilic;

R₂, R₃ and R₄, which may be the same or different, are independently selected from H, halogen, alkyl, alkoxy, acyl, alkenyl, aryl, alkylaryl, arylalkyl, or a silyl group, whether unsubstituted or substituted, optionally interrupted by one or more heteroatoms, straight chain or branched chain hydrophilic or fluorophilic.

R₃ or R₄+R₂ can be a saturated or an unsaturated cycloalkane; and

“═” represents a single bond or a double bond,

or a compound of general formula II

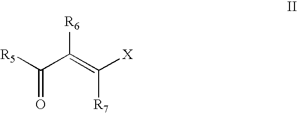

wherein R₅, R₆ and R₇, which may be the same or different are independently selected from H, halogen, alkyl, alkoxy, alkenyl, alkynyl, aryl, arylalkyl, carboxyl, acyl, acyloxy, acylamino, formyl and cyano whether unsubstituted or substituted, optionally interrupted by one or more hetero atoms, straight chain or branched chain, hydrophilic, hydrophobic or fluorophilic and X is a halogen,

wherein the amount of the compound(s) is effective to degrade or cause sloughing of the biofilm.

The use of compounds of formulae I and if result in the eventual loss of the biofilm or make it easier to remove mechanically (eg, by wiping away in the shower/toilet or creating turbulence in fluid in a conduit). These compounds may also increase the susceptibility of the biofilm to traditional biocides (and antibiotics) in addition to helping to remove the biofilm through sloughing processes. Thus, the inclusion of adjunct therapies might have synergistic effects, especially on biofilms and would certainly help in the killing of the newly removed biofilm cells.

The compositions of the third aspect of the invention may be in any suitable form. The composition may include a carrier or diluent. The carrier may be liquid of solid. For example, the compositions may be in the form of a solution of suspension of the compounds in a liquid. The liquid may be an aqueous solvent or non-aqueous solvent. The liquid may consist of or comprise a one of more organic solvents. The liquid may be an ionic liquid. Particular examples of carrier or diluents include, but are not limited to, water, polyethylene glycol, propylene glycol, cyclodextrin and derivatives thereof.

The composition may be formulated for delivery in an aerosol or powder form.

The composition may include organic or inorganic polymeric substances. For example, the compound of formula I or II may be admixed with a polymer or bound to, or adsorbed onto, a polymer.

When the composition is to be formulated as a cleaning formulation, the composition may include conventional additives used in such formulations. Non-limiting examples of the physical form of the formulations include powders, solutions, suspensions, dispersions, emulsions and gels.

Formulations for pharmaceutical uses may incorporate pharmaceutically acceptable carriers, diluents and excipients known to those skilled in the art. The compositions make be formulated for parenteral or non-parenteral administration. The composition of the invention may be formulated for methods of introduction including, but not limited to, topical, intradermal, intramuscular, intraperitoneal, intravenous, subcutaneous, intranasal, epidural, ophthalmic, and oral routes. It may be formulated for administration by any convenient route, for example by infusion or bolus injection, by absorption through epithelial or mucocultaneous linings (e.g., oral mucosa, rectal and intestinal mucosa, etc) and may be administered together with other biologically active agents. Administration may be localized or systemic. The composition may be formulated for intraventricular and intrathecal injection. Pulmonary administration can also be employed, e.g. by use of an inhaler or nebulizer, and formulation with an aerosolizing agent.

In certain preferred embodiments the composition further comprises other active agents such as antibiotics and cleaning agents. As will be understood the degradation or sloughing of the biofilm will allow greater penetration of, for example, antibiotics into the biofilm enabling greater removal of the biofilm.

In a fourth aspect, the present invention provides a method of treating an infection in a human or animal subject in which a biofilm is formed, the method comprising administration to the subject of an effective amount of the composition of the invention.

Biofilms are responsible for disease such as otitis media (inflammation of the inner ear). Other disease in which biofilms play a role include bacterial endocarditis (infection of the inner surface of the heart and its valves), cystic fibrosis (as already mentioned above), and Legionnaire's disease (an acute respiratory infection). The method of the third aspect may be used to treat such medical conditions.

The method may also used to treat biofilm formation resulting from a skin infection, burn infection and/or wound infection. The method and composition of the invention may be particularly suitable for the treatment of infection in immuno compromised individuals.

In yet a fifth aspect, the present invention provides a method for treating a surface to degrade or cause sloughing of at least a portion of the biofilm formed on the surface the method comprising contacting the surface with a compound in accordance with the present invention.

The term “surface” as used herein relates to any surface which may be covered by a biofilm layer. The surface may be a biological (eg tissue, membrane, skin etc) or non-biological surface.

The surface may be that of a natural surface, for example, plant seed, wood, fibre etc.

The surface may be any hard surface such as metal, organic and inorganic polymer surface, natural and synthetic elastomers, board, glass, wood, paper, concrete, rock, marble, gypsum and ceramic materials which optionally are coated, eg with paint, enamel etc; or any soft surface such as fibres of any kind (yarns, textiles, vegetable fibres, rock wool, hair etc.); or porous surfaces; skin (human or animal); keratinous materials (nails etc). The hard surface can be present in a process equipment member of a cooling equipment, for example, a cooling tower, a water treatment plant, a dairy, a food processing plant, a chemical or pharmaceutical process plant. The porous surface can be present in a filter, g. a membrane filter.

Particular examples of surfaces that may be treated in accordance with the invention include, but are not limited to, toilet bowls, bathtubs, drains, highchairs, counter tops, vegetables, meat processing rooms, butcher shops, food preparation areas, air ducts, air-conditioners, carpets, paper or woven product treatment, napples(diapers), personal hygiene products (eg sanitary napkins) and washing machines. The cleaning composition may be in the form of a toilet drop-in or spray-on for prevention and removal of soil and under rim cleaner for toilets. The composition and method of the present invention also have application to cleaning of industrial surfaces such as floors, benches, walls and the like and these and other surfaces in medical establishments such as hospitals (eg surfaces in operating theatres), veterinary hospitals, and in mortuaries and funeral parlours.

In one embodiment, the method comprises the steps of administering a cleaning-effective amount of a furanone compound described above to a biofilm-containing surface or a surface to ensure that it is biofilm-free. In another form of the present invention, one would admininster an amount of the cleaning compound described above effective to prevent biofilm build-up or formation on a surface.

In particularly advantageous forms of the present invention, the method is used to remove biofilm on food preparation surfaces, such as kitchen counters, cutting boards, sinks, stoves, refridgerator surfaces, or on sponges and other cleaning implements, such as mops and wipes.

In another advantageous form of the present invention, the method is used to remove biofilm or bathroom surfaces, such as toilets, sinks, bathtubs, showers, and drains.

In another form, the present invention is used to remove biofilm on clothing and other woven and soft surfaces. This may be by means of a wipe, sponging or soaking method or by a laundering or detergent method. In another form of the present invention, the method is used to remove biofilm on floors and window surfaces, especially surfaces that are exposed to moisture, such as kitchen floor, shower stalls, and food production areas. In another form of the present invention, the method is used to remove biofilm in large-scale sanitation applications, such as food production machinery, processing areas and conduits that carry raw materials or finished products.

The compound of the present invention may be used in the preparation of epidermal bandages and lotions. Alternatively, the compounds of the invention may be incorporated into cosmetic formulations, for example, aftershave lotions.

Compositions of the present invention may be in the form of an aqueous solution or suspension containing a cleaning-effective amount of the active compound described above. The cleaning composition may be in the form of a spray, a dispensable liquid, or a toilet tank drop-in under-rim product for prevention, removal and cleaning of toilets and other wet or intermittently wet surfaces in domestic or industrial environments.

The composition of the present invention may additionally comprise a surfactant selected from the group consisting of anionic, nonionic, amphoteric, biological surfactants and mixtures thereof. Most preferably, the surfactant is sodium dodecyl sulfate.

One or more adjuvant compounds may be added to the cleaning solution of the present invention. The may be selected from one or more of biocides, fungicides, antibiotics, and mixtures thereof to affect planktonics, pH regulators, perfumes, dyes or colorants may also be added.

By “cleaning-effective” amount of active compound, it is meant an amount of the compound which is necessary to remove at least 10% of bacteria from a biofilm as determined by a reduction in numbers of bacteria within the biofilm when compared with a biofilm not exposed to the active compound.

The cleaning methods of the present invention are suitable for cleaning biofilm deposits. They may be used to treat hard, rigid surfaces such as drain pipes glazed ceramic, porcelain, glass, metal, wood, chrome, plastic, vinyl and formica or soft flexible surfaces such as shower curtains, upholstery, laundry and carpeting. It is also envisioned that both woven and non woven and porous and non-porous surfaces would be suitable.

In other embodiments of the present invention, the composition of the invention may be formulated as a dentifrice, a mouthwash or a composition for the treatment of dental caries. The composition may be formulated for acne treatment or cleaning and disinfecting contact lenses (eg as a saline solution).

The method of the invention may be used to treat biofilms on implanted devices that are permanent such as an artificial heart valve or hip joint, and those that are not permanent such as indwelling catheters, pacemakers, surgical pins etc. The method may further be used to remove biofilm in situations involving bacterial infection of a host, either human or animal, for example in a topical dressing for burn patients. An example of such a situation would be the infection by P. aeruginosa of superficial wounds such as are found in burn patens or in the lung of a cystic fibrosis patient.

In other forms, the present invention can be used to treat biofilms developing in the process of manufacturing integrated circuits, circuit boards or other electronic or microelectronic devices.

Throughout this specification the word “comprise”, or variations such as “comprises” or “comprising”, will be understood to imply the inclusion of a stated element, integer or step, or group of elements, integers or steps, but not the exclusion of any other element integer or step, or group of elements, integers or steps.

All publications mentioned in the specification are herein incorporated by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

As indicated above, the method and composition of the present invention have application to any biofilm. The following discussion provides a possible explanation for the effectiveness of the compounds of the invention in causing sloughing of one particular category of biofilm forming microorganisms. However, it is to be understood that the invention is not to be limited by this explanation or to the particular microorganisms described below.

Many host-associated bacteria use chemical signals to monitor their own species population density and to control expression of specific genes in response to population density. This type of gene regulation is termed quorum sensing (Fuqua et al., 1997) and is a generic phenomenon described in many Gram-negative (Eberg, 1999; Greenberg, 1997) and Gram-positive bacteria (Kleerebazem et al., 1997). Many Gram-negative bacteria capable of quorum sensing employ acylated homoserine lactones (AHL) as the signalling compound. The various AHL compounds described in Gram-negative bacteria differ between one another in length and substitutions on their acyl side chains. The signalling molecule is synthesized by a LuxI-type synthase and they bind to a cognate LuxR-type transcriptional activator protein to regulate-expression of target genes. At low cell density, the signalling compound is synthesized at a low basal level and is thought to diffuse into the surrounding media where it becomes diluted. During growth, the AHL accumulates in the medium until a critical threshold concentration is reached. At this concentration, the AHL binds to its cognate receptor, which in turn becomes activated and stimulates or represses transcription of target genes.

Pseudomonas aeruginosa, a Gram-negative opportunistic human pathogen, is responsible for persistent and often incurable infections in immunocompromised people and individuals with cystic fibrosis (Hoiby, 2000; Koch & Hoiby, 1993; Pollack, 1990). The list of P. aeruginosa quorum sensing controlled (qsc) genes and phenotypes is continuously growing (Glessner et al., 1999; Hassett et al., 1999) and classes of qsc genes are emerging (Whiteley et al., 1999). For reviews see (Passador & Iglewski, 1995; Pesci & Iglewski, 1997; Swift et al., 1996; Van Delden & Iglewski, 1998; Williams et al., 2000).

Two AHL-mediated quorum sensing circuits has been identified in P. aeruginosa. The las system consists of lasI, an AHL synthase gene responsible for the synthesis of OdDHL (N-[3-oxo-dodecanoyl]-L-homoserine lactone; 3-oxo-C12-HSL; PAJ-1) (Pearson et al., 1994), and lasR that encodes a LuxR-type transcriptional regulator protein (Gambello & Iglewski, 1991; Passador et al., 1993). The las system has been shown to regulate the expression of several virulence factors such as extracellular enzymes (LasB elastase, LasA protease, akaline protease), secondary metabolites (pyocyanin, hydrogen cyanide, pyoverdin), toxins (exotoxin A), and lasI itself In the rhl system, the rhlI gene product direct the synthesis of BHL (N-butanoyl-L-homoserine lactone; C4-HSL PAI-2), which in conjunction with the rhlR gene product activates transcription of the rhlAB rhamnolipid biosynthesis genes and the rdlI gene itself. The rhl system is also involved in modulating the expression of several of the virulence factors controlled by the las system (Glessner et al. 1999; Pearson et al., 1995).

In the CF lung, P. aeruginosa grows primarily as biofilms (Hoiby, 1977, Lam et al., 1980; Singh et al., 2000), which provides protection from the host defence system and from the action of antibiotics (Koch & Hoiby. 1993). Biofilms are highly structured, surface-attached communities of cells enclosed in self-produced polymeric matrix. In laboratory-based systems, P. aeruginosa forms biofilms several hundred micrometers thick with tower- and mushroom-shaped microcolonies intervened by water channels and void spaces (Costerton et al., 1995; Davies et al.. 1998). The current model is that biofilm formation proceeds through a series of programmed events. O'Toole and Kolter (1998) have demonstrated that flagellar mobility and type IV pili-mediated twitching mobility in P. aeruginosa is necessary for surface attachment and colonization. There is compiling evidence that cell-to-cell communication plays a crucial role for the maturation of biofilms, ie. for the development of a characteristic three-dimensional biofilm architecture. For P. aeruginosa it has been demonstrated that the ability to form biofilms in flow chamber systems is affected by the las but not the rhl quorum sensing system (Davies et al., 1998). While the wild-type formed characteristic microcolonies separated by water channels, the last mutant developed only flat undifferentiated biofilms, which exhibited greater sensitivity to the biocide sodium dodacyl sulfate. These results argue in favor of final overlaps between factors necessary for cell-to-cell signalling, biofilm maturation and bacterial pathogenesis.

Phenotypes regulated by cell-to-cell communication have been proven or suggested to be important for bacterial colonization of eukaryotes (Ebert et al., 1996; Givskov et al. 1996; Kjellaberg et al., 1997; Piper et al., 1993: von Bodman & Farrand, 1995; Givskov et a., 1996). Given the widespread occurrence of AHL-mediated cell-to-cell communication systems, it has been hypothesized that higher organisms may have evolved specific means to interfere with bacterial communication and possibly escape colonization. The Australian marine macroalga Detisea pulchra has been suggested to possess such a countermeasure to bacterial processess (Kjelleberg et al., 1997). The alga produces a number of halogented furanones (de Nys et al., 1993; Relchelt & Borowitzka, 1984) which display strong bacterial activities, including antifouling and antimicrobial properties (de Nys et al., 1995; Relchelt & Borowiztka, 1984). Most interestingly, recent reports indicate that some furanones possess AHL-antagonistic activity, which likely can be attributed to a structural similarity to AHLs (Givskov et al. 1996; Manefield et al., 1999, Manefield et al., 2000).

### BRIEF DESCRIPTION OF FIGURES

FIG. 1. Schematic drawings of lasB reporter fusions (not to scale). (A) lasB-gfp(ASV) translational fusion vector pMHLB. (B) pMHLAS with lasB fusion and lasR expressed from the lac promoter. (C) gfp expression cassette of pMH306. (D) L-arabinose controlled gfp(ASV) expression cassette of pBADGfp. The indicated NotI fragments are maintained an a Pseudomonas-shuttle vector of the pUCP-series and an the mini-Tn5 delivery vector, pTn5-Grn. The genetic components are: PlasB, elastase (LasB) promoter fragment gfp(ASV), gene encoding the unstable Gfp(ASV); T₀, transcriptional terminator from phage lambda, T₁, transcriptional terminator from rmB operon of E. coli, P_{A1/O}, a strong, synthetic LacI-repressible promoter; RBSII, synthetic ribosome binding site; araC P_{BAD}, the promoter of the E. coli araBAD operon and the gene encoding the positive and negative regulator of this promoter, araC.

FIG. 2. Characterization of lasB-based quorum sensing reporter. (A) Induction of pMHLAS in E. coli MT102 by different AHL compounds, all at 1000 nm. The relative green fluorescence emitted by the cells was calculated as the fluorescence at 515 nm divided by the optical density at 600 nm. The AHL compounds assayed were: OdDHL (N-[3-oxo-dodecanoyl]-L-homoserine lactone), ODHL (N-[3-oxo-decanoyl]-L-homoserine lactone), DHL (N-decanoyl-L-homoserine lactone), OOHL (N-[3-oxo-octanoyl]-L-homoserine lactone), OHL (N-octanoyl-L-homoserine lactone). OHHL (N-[3-oxo-hexanoyl]L-homoserine lactone). HHL (N-hexanoyl-L-homoserine lactone). BHL (N-butanoyl-L-homoserine lactone). The results are mean ±SEM of three independent experiments. (B) OdDHL-mediated induction of the PlasB-gfp(ASV) Plac-lasR reporter cassette on a mini-Tn5 transposon integrated into the chromosome of PAO-JP2. The results are mean ±SEM of three independent experiments. (C) Phase contrast and epifluoroscence microphotographs of OdDHL-induced PAO-JP2 cells containing the mini-Tri5-based reporter system. The OdDHL concentrations used were: (I) 10 nM, (II) 100 nM, and (III) 1000 nM.

FIG. 3. Inhibition of quorum sensing by furanone 56. (A) Molecular structure of furanone 56 (MVW: 175 g/mol). The asterisk indicates position 3 on the furanone ring, (B) Response of PAO-JP2 mini-Tn5-PlasB-gfp(ASV) Plac-lasR to OdDHL and furanone 56. The fluorescence signal has been normalized to 100% for 100 nm OdDHL and 0 μg/ml furanone 56. (C) Induction of the mini-Tn5-based PlasB-gfp(ASV) reporter in wild type P. aeruginosa PAO1 in the presence of: (●) 0 μg/ml furanone 56, (σ) 5 μg/ml furanone 56, (V) 10 μ/ml furanone 56. (▪) PAO1 with the pMH391 vector control. (D) Growth of P. aeruginosa PAO1 in the presence of furanone 56. Symbols as in (C).

FIG. 4, P. aeruginosa PAO-JP2 virulence factor production in the presence of OdDHL and furanone 56. (A) Elastase activity. (B) Chitinase activity.

FIG. 5. Inhibition of OdDHL-mediated signalling in P. aeruginosa biofilm. Twenty-four hours old biofilms of P. aeruginosa PAO-JP2 carrying the mini-Tn5-based PlasB-gfp(ASV) Plac-lasR reporter were established in flowcells. The medium was switched to contain: (I) 40 nm OdDHL, (II) 40 nm OdDHL and 2 μg/ml furanone 56, and (III) 80 nm OdDHL and 2 μg/ml furanone 56. Prior to the switch (0 h), the microscope was programmed to track selected microcolonies. Reflection and epifluorescence images were recorded by CSLM during the 8 hours on-line experiment. The scalebar is 20 μm.

FIG. 6. Cell-density dependent activation of the PlasB-gfp(ASV) reporter in P. aeruginosa PAO1 biofilm. Green fluorescence indicates active transcription of the quorum sensing controlled lasB gene. The bacteria constitutively express Rfp to visualize the biomass at the substratum (right panel). Simulated fluoroscence projections generated by CSLM after (I) 12 h and (II) 48 h post-inoculation. The scalebar is 20 μm.

FIG 7. Effect of furanone 56 on wild type P. aeruginosa quorum sensing and biofilm formation. P. aeruginosa PAO1 carrying the lasB-based reporter and a dsred expression cassette on mini-Tn5 transposons was cultivated in flowcells in the absence or presence of 5 μg/ml furanone 56. In the simulated fluorescence projections generated by CSLM, green fluorescence indicates active transcription of the quorum sensing controlled lasB promoter. Red fluorescence arises from constitutive expression of the dsred gene and, therefore, correlates to bacterial biomass accumulation at the substratum. Single cells may emit both green and red fluoroscence but, for clarity, the colours are shown in separate images. The lower images provide saggital views to visualize biofilm structure and thickness (day 7). The scalebar is 20 μm.

FIG. 8. Effect of furanone 56 on the V. fisheri lux quorum sensing system In P. aeruginosa background. The plasmid pJBA132Gm carrying the luxR luxI-gfp(ASV) repoter (Andersen et al., 2001) was transferred to PAO-JP2. The resulting strain, PAO-JP2(pJBA312Gm), was grown in flowcells and studied by CSLM. A 24 hours old, non-fluorescent biofilm (A) was exposed to 250 nM OHHL. Within one hour biofilm bacteria became green fluorescent (B). The medium was then further modified to contain 250 nM OHHL and 15 μg/ml furanone 56. After an additional 2 hours, biofilm bacteria were significantly less green fluorescent (C). Six hours following the introduction of furanone 56, green fluorescence had almost completely disappeared (D). The scalebar is 20 μm.

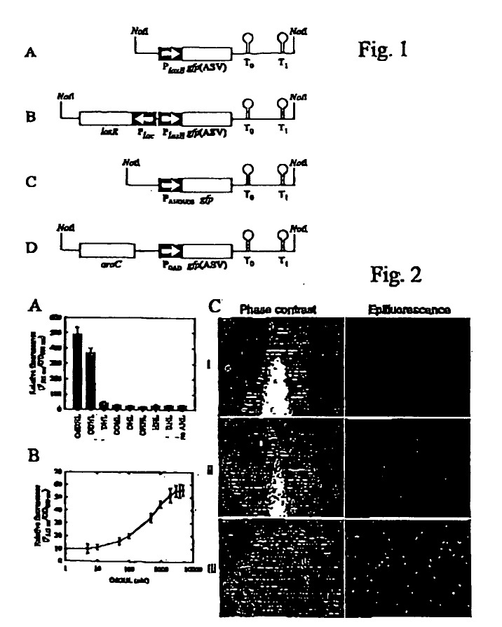

### DETAILED DESCRIPTION

In order that the nature of the present invention may be more clearly understood preferred forms thereof will now be described with reference to the following non-limiting examples.

Materials and Methods

Bacterial Strains. Escherichia coli and P. aeruginosa strains used in this study are listed in Table 2.

Media. The basic medium was either modified Luria-Bertani (LB) medium (Bertani, 1951) containing 4 g/liter of NaCl or ABt minimal medium (AB minimal medium (Clark & Maaloe, 1967) containing 25 mg/liter of thiamine). Antimicrobial agents were added as appropriate at the following concentrations: Gentamycin, 15 μg/ml for E. coli and 60 μg/ml for P. aeruginosa; ampicillin, 100 μg/ml for E. coli; carbenicillin, 300 μg/ml for P. aeruginosa; tetracycline. 60 μg/ml for P. aeruginosa.

Plasmids and DNA manipulations. The plasmids used in this study are listed in Table 2 DNA treatment with modifying enzymes and restriction endonucleases (GiboBRL Life Technologies, Rockville, Md., USA), ligation of DNA fragments with T4 ligase (GibcoBRL Life Technologies, Rockville, Md., USA), and transformation of E. coli were performed using standard methods (Sambrook et al., 1989). Plasmid DNA was isolated with a Spin Miniprep kit (Qiagen, Hilden, Germany) and DNA fragments were excised and purified from agarose gels using GFX DNA and Gel Band Purification kit (Amersham Pharmacia Biotech, Piscataway, N.J.). Polymerase chain reaction was carried out on a Biometra T3 thermocycler using Expand High Fidelity PCR kit (Boehringer Mannheim, Germany). Transformation of P. aeruginosa was performed accordingly to a previously described method (Diver et al., 1990).

The transcriptional fusion vector pMH391 was constructed by inserting the 1765-bp NotI fragment containing the RBSII-gfp(ASV)-T0-T1 cassette of pJBA25 (J. B. Andersen, unpublished) into NotI-digested pUCP22Not. A translational fusion between the NH-terminal part of lasB and an unstable variant of the gfp gene was constructed. The first codon of the lasB gene was maintained and fused to the gfp(ASV) open reading frame devoid of the start codon (Andersen et al., 1998). The fusion retains the lasB promoter and the 5′ untranslated region of the lasB transcript and ensures that the native RBS and the spacing to the start codon is preserved and therefore, that the activity of the reporter gene fusion closely reflects the expression of the lasB gene. The quorum sensing reporter system. pMHLAS, was constructed by a two-step cloning procedure. The PlasB-gfp(ASV) translational fusion was made by amplifying a 348-bp PCR product starting 345-bp upstream of the lasB initialization codon, using the primers lasB fwd and lasB rev and chromosomal DNA of P. aeruginosa PAO1 as template. The PCR-fragment was subsequently digested with XbaI and SphI and inserted into the corresponding site of pMH391. This gave rise to the plasmid pMHLB, which carries the translational PlasB-gfp(ASV) followed by translational stop codons in all three reading frames and two strong transcriptional terminators (Andersen et al.. 1998).

In order to enhance the sensitivity of the quorum sensing monitor, the lasR gene under control of the lac promoter was inserted upstream of the PlasB-gfp(ASV). The lac promoter was chosen to drive lasR expression since previous studies have demonstrated that lasR under its own promoter was insufficient to activate the lasB promoter in the presence of OdDHL (Pearson et al., 1995). The presence of lasR on the monitor plasmid allows use of very sensitive E. coli-based monitor stains harboring the construct in high copy-numbers. A 1002-bp BamHI fragment containing the Plac-lasR expression cassette was generated by PCR amplification with the primer set lasR fwd and lasR rev and with pKDT17 as template. The fragment was inserted into the unique BamHI site of pMHLB. The resulting plasmid, pMHLAS, contained divergent transcribed Plac-lasR and PlasB-gfp(ASV) fusions on a 312-bp fragment flanked by NotI restriction sites.

The NotI cassette was excised from pMHLAS and inserted into the unique NotI site of the pTn5-Gm vector to create pTn5-LAS.

The araC-P_{BAD} controlled gfp(ASV)-expression vector was constructed by PCR amplification of a 1658-bp fragment containing the araC-P_{BAD} region using the primers araCP fwd and araCP rev and pBAD18 as template. The araC-P_{BAD} fragment was digested using the restriction endonucleases BclI and XbaI and was then ligated into the BamHI-XbaI site of pMH391 giving rise to pBADGfp. The araC-P_{BAD}gfp(ASV) cassette was subsequently excised as a NotI fragment and moved into the corresponding site of pTn5Gm to give pTn5-BADGfp.

The plasmid used to provide a red fluorescent color-tag on bacteria was constructed as follows. pDsRed was digested with NotI, polished with T4 DNA. Polymerase, and digested with PvulI. A 916 bp blunt-ended fragment containing the dsred gene under the lac promoter was isolated and inserted into the blunt-ended EcoRI-HindIII site of pMH391. This resulted in pMH210 with Plac-dsred followed by translational stop codons in all three reading frames and two strong transcriptional terminators. The dsred-expression cassette was excised as a 1916 bp Not-fragment and moved into the corresponding site of the pUTTc delivery vector to yield pTn5-Red. The lac promoter of E. coli acts as a constitutive promoter In Pseudomonas spp. due to the absence of lac repressor activity (Andersen et al., 1998).

The reporter cassettes were inserted at random positions in the chromosomes of P. aeruginosa PAO1 and PAO-JP2 by triparental mating. The selected transconjugants with random insertion of the mini-Tn5 elements showed no sign of phenotypic changes compared to the parental strains, when tested in liquid medium or flow-chamber biofilms.

AHL and furanone bioassay. Strains were grown exponentially in LB or ABt medium supplemented with 0.5% glucose at 30° C., shading at 250 rpm. At an optical density of approximately 0.8. the cultures were diluted and split into subcultures in glass culture flasks. AHLs and furanone 56 were added to appropriate concentrations and the cultures were further incubated at 30° C. under vigorous shaking. Culture samples were retrieved at various time intervals and green fluorescence was measured with a fluorometer (model RF-1501, Shimadzu, Tokyo, Japan) set at an excitation wavelength of 475 nm and emission wavelength of 515 nm. Relative fluorescence was calculated as green fluorescence normalized to 1 ml culture divided by the optical density (OD_{600 nm}.

P. aeruginosa biofilms. Biofilms were grown at 30° C. in three-dimensional flow cells (Christensen et al., 1999) with individual channel dimensions of 0.3×4×40 mm supplied With ABt minimal medium supplemented with 2% LB. The flow system was assembled and prepared as described by Christensen et al. (1999). The substratum consisted of a microscope glass coverslip (Knittel 24×50 mm st1, Knittel Glaser, Braunschweig, Germany). Cultures for inoculation of the flow channels were prepared in the following way: P. aeruginosa strains were streaked on LB plates with the appropriate antibiotics and incubated for 24 h at 37° C. From each plate a single colony was used for inoculation of 10 ml ABt with 10% LB. The cultures were grown at, 30° C. for 18 h before they were diluted to an OD_{600 nm} of 0.1 in sterile 0.9% NaCl and used for inoculation of the flow channels. Medium flow was kept at a constant rate of 3 mL/h, equivalent to a mean flow velocity of 0.7 mm/s, using a Watson-Marlow 205S peristaltic pump (Watson-Marlow, Falmouth, England). Biofilms were grown for 24 hours before being shifted to media containing AHL and furanone.

Measurements of virulence factors. PAO-JP2 was grown in LB medium at 37° C. and shaking at 250 rpm to an OD_{600 nm} of 1.0. The culture was divided into seven subcultures, which were added 0, 70, or 1000 nm OdDHL and 0. 3. or 5 μg/ml furanone 58. The cultures were grown for an additional 4 hours at 37° C. The proteolytic activity was measured as described by Ayora & G\overline{o}tz (1994). Azocasein (250 μl 2%, Sigma, St. Louis, Mo.) in 50 mM Tris/HCl and steril-filtered (Ø 0.2 μm) supernatant (150 μl) were incubated for 4 h at 4° C. After precipitation of undigested substrate with trichloroacetic acid (1.2 ml 10%) for 15 minutes, followed by 10 minutes centrifugation at 1000 rpm, NaOH (1.4 ml 1 M) were added to the supernatant. The relative protease activity was measured as the absorbance at 440 nm (OD_{440 nm}) of the supernatant divided by the optical density of the culture (OD_{600 nm} ).

The chitinase activity assay was performed as described by the assay manufacturer (Loewe Biochemica, Sauerlach, Germany). Supernatant (560 μl) of cultures prepared as described for the elastase assay was mixed with carboxymethyl-chitin-remazol brilliant violet (200 μl) and sodium phosphate buffer (40 μl, 1 M, pH 7.5). The reaction mixture was incubated for 18 h at 40° C. in a waterbath. The reaction was stopped by addition of HCl (200 μl, 2 N) and kept for 15 min on ice. After centrifugation (10 min at 15000 rpm), the absorbance (OD_{550 nm}) of the supernatant was measured. The relative chitinase activity was calculated as OD_{550 nm}/OD_{600 nm} normalized to 1 ml supernatant.

Scanning confocal laser microscopy (SCLM). Microscopic inspection and image acquisition were performed an a scanning confocal laser microscope (model TCS4D, Leica Lasertechnik GmbH, Heldelberg, Germany) equipped with a 63×/1.32-0.6 oil objective. The microscope was equipped with a motorized and programmable xy-stage, which was used for monitoring single colonies during the biofilm experiments. At the beginning of each online experiment, the microscope was programmed to track single randomly selected microcolonies; the sensitivity of photo multipliers and the laser intensity were adjusted and thereafter kept constant through out the duration of the experiments. Image scanning was carried out using the 488 nm and 568 nm lines of an Ar/Kr laser for detection of Gfp and Rfp, respectively. Visualization of captured images was performed using the IMARIS software package (Bitplane AG, Zúrich, Switzerland) running on a Silicon Graphics Indigo 2 workstation (Silicon Graphics, Mountain View, Calif. USA).

Results

Construction and characterization of lasB-based AHL monitor. Our genetic construct for detection of AHL signal molecules relies on the availability of a promotor, which is transcriptionally controlled by an AHL-activated LuxR-type receptor protein. Several target genes of the las and rhl quorum sensing systems of P. aeruginosa have been identified (Oschner & Reiser, 1995; Passador et al., 1993; Pearson et al., 1997; Whiteley et al., 1999; Winson et al., 1995). For the purpose of OdDHL detection, we have chosen the well-characterized and tightly regulated lasB promotor. Several regulatory elements of the lasB promotor such as pulative regulatory sequences have been described (Anderson et al., 1999; Fukushima et al., 1997; Gray et al., 1994; Rust et al., 1996). Previous studies using a PlasB-lacZ transcriptional fusion in E. coli MG4 have demonstrated a 63-fold induction of the promotor in response to OdDHL addition (Gray et al., 1994).

We have constructed a reporter system consisting of a translational fusion of the lasB promotor to a gene encoding a unstable variant of Gfp, Gfp(ASV) (Andersen et al., 1998). Expression of the reporter is controlled by LasR from P. aeruginosa in conjunction with OdDHL. Several plasmid-based systems which feature high as well as low copy numbers have been used to accommodate the present reporter cassette in P. aeruginosa. These include pUCP-series of Pseudomonas-shuttle cloning vectors (Bloemberg et al., 1997; West et al., 1994), the segregationally stable pME6030based vectors (Heeb et al., 2000), and mini-Tn5 transposon systems for chromosomal integration (de Lorenzo et al., 1990). The copy number of each system in P. aeruginosa is 10, 2-4 , and 1, respectively (de Lorenzo et al., 1990: Heeb et al., 2000; Schweizer, 1991).

Initially, the lasB-gfp(ASV) translational fusion (OdDHL sensor) was thoroughly characterized with respect to its sensitivity and specificity. A culture of E. coli hosting the pMHLAS monitor plasmid was diluted and spilt into several subcultures which were then supplemented with AHLs at concentrations ranging from 0 to 1000 nM. Not surprisingly, the most efficient inducer of the monitor was OdDHL, the cognate signal molecule of the las quorum sensing system. A closely related analog, ODHL (3-oxo-C10-HSL), also activated lasB-expression albelt at a lower level. The remaining AHL compounds did not induce significant expression of the reporter gene at a concentration of 1 μM (FIG. 2a). When the pMHLAS based reporter system was hosted by PAO-JP2. the OdDHL concentration required for half-maximal activation of the lasB-gfp(ASV) fusion was 8 nM (data not shown). In single copy on the chromosome of PAO-JP2, the OdDHL concentration on required for half-maximal activation of lasB expression was approximately 250 nM (FIG. 2b). Green fluorescent cells were visible by epifluorescence microscopy at a minimal OdDHL concentration of 50 nM (FIG. 2c).

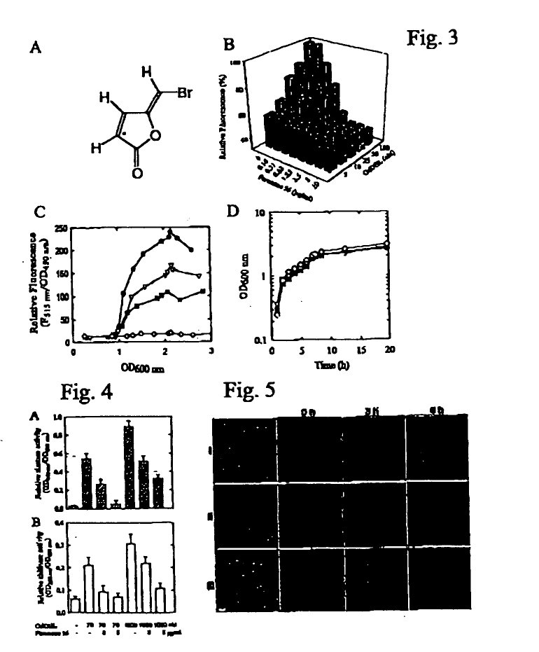

Furanone-mediated inhibition of quorum sensing. Furanone compounds produced by the Australian macroalga D. pulchra have been shown to possess quorum sensing inhibitory (QSI) properties as well as interfering with complex surface-dependent phenomenons such as swarming motility and biofilm formation of Serratia liquefaciens (Givskov et al., 1996; Undun et al., 1998; Manefield et al., 1999; Manefield of al. 2000). Natural furanone compounds have a rather limited effect on P. aeruginosa (data not shown). However, natural QSI compounds can be further modified by means of combinational chemistry which is a highly efficient method to generate a large number of analogues for screening purposes. One such synthetic furanone compound, termed furanone 56, is characterized by a lack of side chain at the position 3 on the furanone ring. This compound only contains one bromine substitution at the methylene group and no bromine substitution on the furanone ring (FIG. 3a).

To investigate whether the furanone compound efficiently inhibited the las quorum sensing system, plankonic cultures of PAO-JP2 cells harboring the PlasB-gfp(ASV) reporter were subjected to a range of furanone 56 and OdDHL concentrations. At a concentration of 1.25 μ/ml (7.1 μM) furan 56 inhibited lasB-gfp(ASV) expression at a wide range of OdDHL concentrations (FIG. 3b). In the presence of 100 nM OdDHL about 2 μp/ml (11.4 μM) furanone 56 was required to reduce fluoresence by more than 50%. However, complete inhibition was not attained at any of the tested concentrations. Noteworthy, the inhibitory effect of furanone 56 was relieved at increased concentrations of OdDHL. These results clearly demonstrate that lasB-gfp(ASV) expression is stimulated by OdDHL, while furanone 56 antagonizes this activation.

The PlasB-gfp(ASV) reporter was inserted into the chromosome of wild type P. aeruginosa. Expression of lasB-gfp(ASV) expression was followed along the growth curve in the presence of furanone 56 FIG. 3c shows that lasB-gfp(ASV) expression was induced in a cell density-dependent manner. The quorum size for lasB-gfp(ASV) induction corresponded to a cell density slightly above OD_{800 nm} of 1.0, which is in agreement with other reports (Brumilk & Storey, 1992). The data show that 5 μg/ml (28.5 μM) furanone caused a 40% reduction in lasB(ASV) expression in wild type P. aeruginosa; 10 μg/ml furanone caused a 60% reduction.

To determine if the furanone compound worked specifically on the las quorum sensor and not indirectly by disruption of primary metabolic functions, we followed growth as optical density of P. aeruginosa PAO1 in the presence of furanone 56. FIG. 3d shows that the furanone in concentrations used for this study had no or only little effect on growth. A similar assay with P. aeruginosa PAO-JP2 showed no effect on growth rate (data not shown). Further, we tested whether the furanone affected P. aeruginosa protein synthesis. The wild type P. aeruginosa PAO1 strain containing the consitutive Gfp-expression vector pMH306 was grown in the presence of furanone 56 at concentrations from 0 to 10 μ/ml. Gfp-expression (fluorescence/OD_{600 nm}) was throughout the growth cycle unaffected by the presence of furanone compound (data not shown).

Effect of the furanone in a heterologous background. The direct regulation exerted by the las regulon on lasB expression is well described by numerous studies (Gambello & Iglewski, 1991: Pearson et al., 1994; Pearson et al., 1997). Regulatory complexity is added by the observation that the las quorum sensing circuit itself is subject to global regulators (Albus et al., 1997; Whiteley et al., 2000) and that lasB expression is also controlled by other regulators than LasR (Brumlik & Storey, 1992; Pesci et al., 1999; Schictman et al., 1995). It was, therefore, important to rule out that QSI effect, observed is not caused by furanone interaction with high levels of control. Since there is no AHL-based quorum sensing system present in Escherichia coli (Williams et al., 2000), this bacterium provides an unbiased and well-defined genetic background for studying the direct effects of the furanone on the P. aeruginosa las quorum sensing system. We repeated the above-described experiments using E. coli MT102 as a heterologous host for the reporter system. The QSI activity of the furanone was observed in this background as well (data not shown). The E. coli strain harboring the lasB reporter showed increased responsiveness to OdDHL (approx. 10-fold, see FIG. 2). This is likely to be attributed to the increased copy number of the reporter plasmid. The furanone had no effect on growth of E. coli MT102 (data not shown).

Effect of furanone 56 on virulence factor production. The data presented above utilize a translational reporter fusion to the lasB promotor to study the effect of OdDHL and furanone 56. An obvious limitation to this approach is the restriction of analysis to the level of transcription. We therefore investigated the effect of the furanone directly on production of the qsc virulence factors elastase and chitinase (Passador et al., 1993; Winson et al., 1995). FIG. 4 demonstrates that 70 nm OdDHL induced elastase and chitinase activity in P. aeruginosa PAO-JP2. Addition of 3 μg/ml and 5 μg/ml furanone 56 leads to a reduction of elastase and chitinase activity close to the uninduced level. The activity is entirely restored by the addition of 1 μM OdDHL.

Inhibition of AHL-mediated signalling in P. aeruginosa biofilms. The lasB-gfp(ASV) reported was integrated into the chromosome of PAO-JP2 to ensure stable segregation and a constant gene dosage of the reported system. The strain was growth in flowcells for 24 hours in ABt-LB medium and a 10-15 μm thick biofilm developed. The media flow was subsequently switched to ABt-LB medium containing the appropriate AHL and furanone concentrations. The development of green fluorescence was monitored online by CSLM for 8 hours. FIG. 5 shows that the microcolonies were non-fluorescent prior to switch of media. When switched to medium containing 40 nm OdDHL, expression of the lasB-gfp(ASV) reporter fusion was induced and visible in the single cells within four hours. Switching to a medium containing 40 nM OdDHL and 2 μl/ml furanone 56 did not lead to induction of green fluorescence in the time course of the experiment. However, green fluorescence was induced by 80 nM OdDHL and 2 μg/ml furanone 56. Induction of green fluorescence was established in medium contained 80 nM OdDHL and 4 μg/ml furanone (data not shown) but was observed in the presence of 150 nM ODDHL and 4 μg/ml furanone. No green fluorescence was observed in the presence of 4 μg/ml alone (data not shown).

To determine if the furanone-mediated inhibition of green fluorescence is due to subtle non-specific effects on protein synthesis when the bacteria are growing in a biofilm, we examined expression of green fluorescence in cells harboring the araC-P_{BAD}-gfp(ASV) cassette induced by suboptimal levels of L-arabinose is (0.2%). The cells became green fluorescent within 2 hours after induction. The presence of furanone 56 at concentrations below 10 μg/ml had no effect on Gfp-expression (data not shown).

Furanone 66 represses lasB-expression in wild type P. aeruginosa biofilms. Wild type P. aeruginosa (PAO1) carrying a chromosomally integrated lasB-gfp(ASV) reporter system was grown in flowcells similar to the PAO-JP2-based reporter strain. We focused on studying the effect of furanone 56 in loan term biofilm experiments. In favor of this approach is the observation by Davies et al. (1998) that quorum sensing is involved in maturation of P. aeruginosa biofilms (up to 2 weeks old). In order to support such longterm cultivation, the biofilm medium was modified to contain 0.3 mM glucose instead of 2% LB as a carbon source. In addition, the recently available Red Fluorescent Protein (Rfp) derived from the indopacific sea anemone Discosome was employed to provide a red fluorescent tag on the biofilm bacteria. A mini-Tn5 transposon with the dsred gene under control of the strong constitutive lac promoter was inserted into the chromosome of PAO1 containing the lasB reporter system.

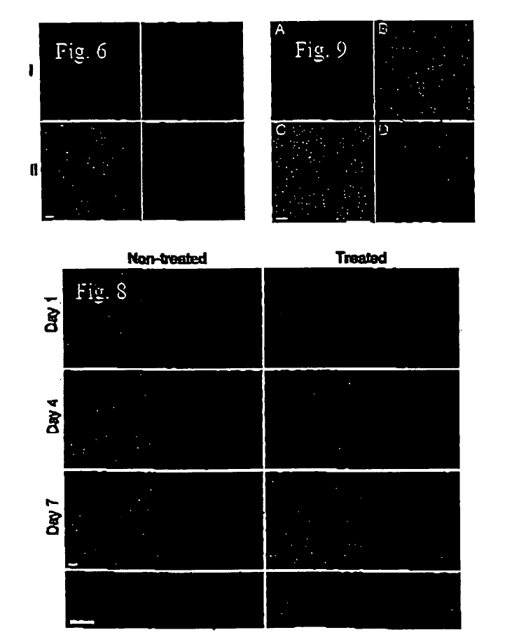

The dual-labeled PAO1 strain was inoculated A grown in flowcells in the absence and presence of 5 μg/ml furanone 56. The flowcells were inspected daily for ten days and scanning confocal photomicrographs were captured (FIG. 6 & 7). For clarity, the green and red fluorescent signals from the same area of the biofilm are shown separately. Because the cells constitutively express Rfp, the red color correlates with cell mass, whereas the green fluorescence indicates active transcription of the lasB-gfp(ASV) reporter gene in response to on-going bacterial communication. We observed that the lasB-reporter in P. aeruginosa PAO1 was activated in a cell-density dependent manner as small microcolonies did not fluoresce green in contrast to larger microcolonies, which were bright green fluorescent (FIG. 6).

As evident from FIG. 7, early biofilm formation (day 1) is not or only slightly affected by the furanone, though bacterial signaling appeared to be greatly reduced. By day 7, the untreated biofilm had grown to an average thickness of 61±6 μm and bright green fluorescence was emitted by the cells. In contrast, the furanone-treated biofilm was 23±4 μm thick and cells were far less green fluorescence. Complete inhibition of the lasB-gfp(ASV) reporter in all biofilm bacteria by addition of furanone in concentrations, which had no effect growth (<10 μg/ml furanone 56), was not achievable.

Repression of LuxR-activated quorum sensing controlled gene transcription. We speculated that the AHL-antagonstic properties of furanone 56 was specific to the P. aeruginosa las quorum sensing system. To test this, a previously published quorum sensing reporter based on the Vibro fisherf luxR gene and Pluxl-gfp(ASV) (Anderson at al, 2001; Wu et al, 2000) was transferred to PAO-JP2. Biofilms of PAO-JP2, grown as described above, were exposed to 250 nM OHHL (N-[3-oxo-hexanoyl]-L-homoserine lactone) and green fluorescence developed within one hour. Green fluoroscence decreased significantly within two hours and completely disappeared after 7 hours when 15 μg/ml furanone 56 was supplied in the medium flow (FIG. 8),

Discussion

Quorum sensing controlled (qsc) gene expression, i.e cell-density dependent gene regulation, has been shown to be a common phenomenon in many Gram-negative bacteria (Fuqua & Greenberg, 1999. Greenberg 1997; Parsek & Greenberg, 2000). In mast cases, quorum sensing systems control expression of virulence factors and hydrolytic enzymes (for recent reviews see Eberl. 1999: Kevit & Iglewski, 2000). More complex phenotypes are also known to be quorum sensing controlled, including swarming motility of S. liquefaciens which is a specialized flagella-driven movement by which a bacterial community can, in the presence of extracellular biosurfactant, spread as a biofilm over a surface (Eberl at ad., 1998; Ebel et al., 1999; Givskov et al., 1997; Givskov et al., 1998; Rasmussen et al., 2000). Evidence is accumulating that the ability to form surface-associated, structured and co-operative consortla (referred to as biofilms) in many organisms may involve quorum sensing regulation (Costerton et al., 1999; Davies et al., 1998; Eberl et at., 1999). P. aeruginosa has become one of the important model organisms for research in this field. This opportunistic pathogen produces a battery of extracellular virulence factors. The quorum sensing circuits of P. aeruginosa have been demonstrated to exert positive transcriptional control on the majority of genes encoding virulence factors, e.g. (elastase), lasA (staphylolytic protease), toxA (exatoxin A), and aprA (alkaline protease) (Brint & Ohman, 1995; Gambello et al., 1993; Gambello & Iglewski, 1991; Ochsner & Reiser, 1995; Pearson et al., 1995; Seed et al., 1995; Toder at al., 1991), Recent studies estimated that 14% of the P. aeruginosa genes are subjeot to quorum sensing control (Whiteley et al., 1999) and, therby, support the view that quorum sensors are involved in global control of gene expression.

P. aeruginosa has been shown to form organized, surface-attached microbial communities, called biofilms. This trait has been linked to pathogenicity of the organism in relation to pulmonary infections in cystic fibrosis (Hoiby & Koch, 1990; Koch & Hoiby, 1993; Pedersen et al, 1992). The biofilm mode of growth seems to provide the ideal scenario for AHL-mediated quorum sensing. In contrast to the planktonic mode of growth, where signal molecules are likely to become diluted in the medium and carried away by flow, biofilms offer a diffusion-limited environment, which may allow the signal compounds to reach the critical threshold concentration (Charlton et al., 2000). A recent study linked quorum sensing and biofilm development by demonstrating that a lasI mutant is incapable of forming a highly structured wild type-like biofilm (Davies et al., 1998). This observation emphasizes the need for studying quorum sensing in P. aeruginosa at the community level and investigating the interplay, between bacterial communication, biofilm made of growth, and pathogenesis.

Clinical studies have shown that the development of resistance to antibiotics in P. aeruginosa is a serious side-effect of the current anti-pseudomonal treatment (Ciofu et al., 1994) This has encouraged us to engage in the development of novel non-antibiotic, anti-bacterial therapies based on QSI compounds that specifically block bacterial signaling systems. In contrast to the traditional anti-microbial agents, QSI compounds work at concentrations that are well below the minimal inhibitory concentrations. This concept is attractive, since such compounds will not create a selection pressure for development of resistance. Furthermore, bacteria that are insensitive to the OSI compounds because of mutations in the LuxR-type receptor proteins are expected to be unable to signal each other and therefore unable to coordinate their effort. Finally, since the selected QSI's are non-toxic for bacteria at the concentrations used they are not expected to exhibit adverse affects on beneficial bacterial consortia present in the host (for example the gut flora).

In this study we have developed novel molecular tools, which allow in situ detection of N-acyl-homoserine lactone-mediated quorum sensing and quorum sensing inhibition in P. aeruginosa biofilms. Our monitor system relies on a reporter gene fusion to a qsc promoter from P. aeruginosa. We have chosen the well characterized lasB promoter (Bever & Iglowski, 1988; Fukushima et al., 1997; Gambello & Iglewski, 1991; Rust et al., 1996; Toder at at, 1994) and used a translational reporter fusion that retains the 5′ untranslated region of the lasB transcript, the native RBS, and spacing to the translational start. This might be important as the 5′ translated lasB mRNA is involved in post-transcriptional iron control of elastase expression (Brumlik & Storey, 1992 Brumlik & Storey, 1998). A unstable variant of Gfp (Andersen at al., 1998; Andersen et al. 2001) has been used as reporter. This protein is an optimal bacterial reporter for non-invasive, real-time studies of gene expression at the single cell level because no exogenous substrates and cofactors are required, except for trace amounts of oxygen for maturation, an Gfp normally does not interfere with growth of the host (Chalfie et al., 1994). Notably, the unstable Gfp variant allows detection of transient bacterial communication.

The present quorum sensing reporter is highly sensitive, even when present as as a single chromosomal copy, and detects OdDHL at concentrations as low as 20 nM (data not shown). In agreement with the study of Passator et al. (1996), we found that OdDHL was most efficient in stimulating lasB promoter activity whereas ODHL and OOHL were less efficient (FIG. 2). None of the other AHL compounds tested resulted in detectable expression of the reporter gene fusion. The concentration of OdDHL needed for half-maximal activation of the lasB promoter was 250 nM, i.e. about one-twentieth of that found in stationary phase culture fluids of PAO1. Pearson et al (1995) reported that 1 μM OdDHL was required for half-maximal activation of a similar construct. However, our estimate is based on a reporter system in a single chromosomal copy In PAO-JP2 whereas the former study used a plasmid-reporter (pKDT17) in a P. aeruginosa rhlR mutant (PAO-R1). The differences in copy number and strain background could account for the different estimates.

We have cultivated P. aeruginosa strains haboring the quorum sensing reporter in laboratory-based flowcells. Using SCLM, we were able to monitor of quorum sensing in situ at the single-cell level in biofilms. In the present report, we did not perform a detailed study on the induction of the reporter system in wild type biofilms in relation to microcolony quorum size or threshold OdDHL concentration. However, we did observe that the size of the microcolonies did correlate with induction of the lasB-reporter fusion as would be expected (FIG. 6). In PAO-JP2 biofilms, lasB promoter activity could be induced by OdDHL, in concentrations as low as 20 nM.

Furanone compounds produced by D. pulchra have previously been demonstrated to specific interfere with several AHL-regulated bacterial processes without any effect on bacterial growth or general protein synthesis capability (Givskov et al., 1996; Manefield et al. 2000). The current hypothesis is that the furanone compounds antagonize AHLs by competition for the binding site on the receptor protein. Recently, Manefield et al. (1999) showed that halogenated furanones, at the concentrations produced by the alga, are capable of displacing OHHL molecules from the cognate LuxR receptor protein.

In this study we have employed a novel synthetic furanone, which displays enhanced AHL-antaganistic properties and has no or little effect on growth of P. aeruginosa. Quantitative data from planktonic cultures showed that furanone 56 caused a significant reduction in OdDHL-activated expression of a lasB-gfp(ASV) reporter in P. aeruginosa. The interference by the furanone 56 occurs in a competitive fashion, though the stochiometric furanone-to-OdOHL ratio is approximately 400:1. This ratio is in good agreement to study by Kline et al. (1999) using structural analogs of OdDHL as possible agonist and antagonists of OdDHL. The disproportionate ratio probably reflects the well-documented high affinity of LasR for OdDHL (Gray et at., 1994; Passador et al., 1998). This might also explain our failure to achieve complete inhibition of lasB expression by addition non-toxic concentrations of furanones 56. The compound repressed lasB promoter activity in a heterologous E. coli background, which is devoid of an AHL-mediated quorum sensing system. This supports the model that the algal metabolite specifically interferes with AHL-dependent gene transcription at the level of the LasR regulatory protein.

The P. aeruginosa las and rhl quorum sensing circuits are subjected to additional levels of regulation. Transcription of lasR was shown to be positively regulated by the virulence factor regulator (Vfr) protein (Albus et al., 1997) and to be subject to negative regulation by the product of the rsaL gene, which was recently identified downstream of lasR (de Kievit et al., 1999). Production of BHL was shown to be reduced in a P. aeruginosa gacA mutant and a model has been proposed that places GacA upstream of LasR and RhlR (Reimmann et al., 1997). Moreover, recent results suggest that the rhl system is controlled by RpoS, the sigma factor, which is required for general stress response of P. aeruginosa (Whiteley et al., 2000). It might be speculated that the furanone interferes with one or more of these higher-level regulatory circuits. To exclude this possibility, we investigated if the furanone affects a heterologous quorum sensing system hosted by P. aerginosa. The Vibrio flscheri lux quorum sensing system represents a distinct cell-to-cell communication system not amenable to endogenous P. aeruginosa regulators and might be regarded as a “clean” system in P. aeruginosa. In the present study, furanone 56 was observed to interfere with OHHL-LuxR activated expression of a luxR PluxI-gfp(ASV) fusion. This strengthens the hypothesis that the furanone antagonizes AHLs by interaction with the LuxR-type receptors. Secondly, the effect on the luxR-PluxI-gfp(ASV) reporter indicates that furanone 56 has a broad activity in interaction with LuxR-type receptor proteins, i.e. the particular furanone is not limited only to be an antagonist of OdDHL-LasR complex formation in P. aeruginosa but might also be used to interfere with AHL-mediated cell-to-cell communication in other Gram-negative bacteria.

The furanone did not have any significant effect on bacterial growth rates at concentrations below 10 μg/ml. In addition we observed no negative, non-AHL related effects on bacterial protein synthesis when Gfp-expression under control of the araBAD promoter was induced by suboptional levels of L-arabinose. The data are in agreement with previous two-dimensional PAGE analysis demonstrating that furanones have no gross effect on bacterial protein synthesis (Manefield et al., 1999).

The lasB transcription date was complemented by measurements of the production of two quorum sensing controlled virulence factors, elastase and chitinase. In PAO-JP2. OdDHL clearly stimulated elastase and chitinase activity. The activities were reduced to near uninduced levels upon addition of furanone 56. Restoration of near fully induced levels could be achieved by addition of excess amounts of OdDHL.

We have developed a novel dual-labeling methodology to study quorum sensing. In wild-type P. aeruginosa biofilms. P. aeruginosa PAO1 was manipulated to contain the lasB-gfp(ASV) fusion as a green fluorescent reporter of quorum sensing. Additionally, the strain was equipped with a chromosomally integrated Rfp-expression cassette to provide a constitutive red fluorescent color-tag on biofilm bacteria. To our knowledge, this is the first report on utilization of the Red Fluorescent Protein In P. aeruginosa.

Inhibition of AHL-mediated signaling in the wild-type strain represents additional challenges: the AHL concentration can not be controlled, and the reporter system is subject to additional regulation by the rhl quorum sensing system, which works in conjunction to the las circuit to maximize lasB expression (Pearson et al., 1995). Furthermore, the reporter system in the wild-type responds to endogenous and exogenous OdDHL, whereas the PAO-JP2-based reporter strain responds solely to incoming signal molecules. Considering the potential involvement of efflux pumps in transport of furanone compounds, this might be an important difference. Transcription of the lasB promoter was approximately 2-fold reduced in planktonic cultures of PAO1. In biofilms, the reporter system was partially shut down in the presence of 5 μg/ml furanone 56. It is uncertain if the relatively weak reduction of lasB expression would be sufficient to render the wild-type strain significantly less virulent. However, keeping in mind that lasB belongs to the top of the quorum sensing cascade (Latifi et al., 1996; Seed et at. 1996), it is likely that qsc genes located at lower levels in the regulatory hierarchy might be more severely affected as these genes require higher OdDHL concentrations for activation. The observations by Davies et al. (1998) indicate the existence of qsc genes involved in late P. aeruginosa biofilm maturation. Our study shows that early biofilm formation, i.e. attachment to the surface, is not affected by the furanone. However, we observed that the wild type biofilm, when grown in the presence of furanone, failed to mature and showed an architecture that strongly resembled the one of the PAO1 lasI mutant observed by Davies et al. (1998). This leads to the hypothesis that the furanone may inhibit expression of the yet unidentified qsc gene(s) responsible for biofilm maturation.

In the present study we have demonstrated the use of furanone compounds as a OSI compounds. Furanone 51 interferes with OdDHL-dependent transcription of a lasB-gfp(ASV) reporter fusion, reduces extracellular elastase and chitinase activity in PAO-JP2 grown in the presence of OdOHL, and has no or little effect on bacterial growth and protein synthesis. Further, we have demonstrated that the furanone is capable of penetrating the P. aeruginosa biofilm matrix where it interferes with quorum sensing controlled gene expression and, as a consequence, with biofilm maturation.

Quantitative Furanone Inhibition of a the LuxR-PluxI-gfp(ASV) Encode Quorum Sensor

To analyse the AHL antagonist activity of the halogenated furanones we tested the compounds for their ability to inhibit 3oxo-C6-HSL (OHHL) induced LuxR dependent expression of green fluorescent protein (GFP) from a Pgfp(ASV) fusion in the AHL monitor strain E. coli MT102 harbouring pJBA89 (Andersen et al., 2001).

Method:

The medium used is minimal ABT containing 0.5% glucose and 0.5% Casamino Acids. In growing cultures of the AHL monitor strain E. coli Mt102 (pJBA89) encoding hocR and a P-gfp(ASV) fusion, GFP(ASV) is produced immediately upon OHHL addition specifically from the LuxR controlled P-gfp(ASV) fusion gene. The sensitivity is high (responsive to as little as 3 nm OHHL) and due to the instability built into the GFP (ASV) variant there is no background production of green fluoroscence (Andersen et al., 2001). GFP expression from the AHL monitor strain E. coli MT102 (pJBA89) and the control strain MT102 harbouring the lacl^{q}, Pₗₐₛ-gfp(ASV) IPTG inducible expression system pMH197 was quantified in the following way. Overnight cultures were diluted four fold in fresh medium and incubated for one hour at 30° C. while OHHL and furanone compounds in the required concentrations were mixed in the wells of microtiter dishes. Next, the bacterial culture was distributed to the wells of the microtiter dish (100 μl aliquots), mixed with the previously pipetted compounds and further incubated for two hours at 30° C. (see below). For measurements of GFP, the microtiter dishes were placed in a light sealed dark box (UnitOne, Birkeroed, Denmark) and illuminated with a halogen lamp (Intralux (5000-1, Volpi, Switzerland) equipped with a 480/40 excitation filter (P44-001, AF Analysentechnik, T\overline{u}bingen, Germany). Green fluorescent images were captured with a Hamamatsu C2400-47 double intensified CCD camera (Hamamatsu Herrsching, Germany) using a 532/10 emission filter (Melles Griot 03 FIV111, Melles Griot, Irvine, Calif.). A PC computer controlled the camera and the images were saved in 16-bit format (the scale has a resolution of 16 colours) using the ARGUS-50 software (Hamamatsu). When absolute values were required, green fluoroscence was measured on a fluorometer (model RF-1501; Shimadzu, Tokyo, Japan) act at an excitation wavelength of 475 nm and emission detection at 515 nm. To establish a correlation between the colour code of the ARGUS-50 captured images and green fluorescence, both colour and relative fluorescence units (RFU), were determined for a dilution series of an E. coli MT102 (pJBA89) culture which had been incubated with 100 OHHL. The dilution giving 14 colours and 520 RFU was defined as having 100% RFU. Accordingly, the other colours were assigned a RFU value and a standard curve-relating colour to RFU was constructed. The best straight line was y=8×−16 where y is RFU and x the number of colours (x must be in the range 2-14 colours for reliable measurements).

| | ROW 1 | | ROW 2 | | ROW 3 | | ROW 4 | | ROW 5 | |
|---|---|---|---|---|---|---|---|---|---|---|
| Well A | 5 | nM OHHL | 10 | nM OHHL | 25 | nM OHHL | 50 | nM OHHL | 100 | nM OHHL |
| | 10 | μg/mL furanone | 10 | μg/mL furanone | 10 | μg/mL furanone | 10 | μg/mL furanone | 10 | μg/mL furanone |
| Well B | 5 | nM OHHL | 10 | nM OHHL | 25 | nM OHHL | 50 | nM OHHL | 100 | nM OHHL |
| | 5 | μg/mL furanone | 5 | μg/mL furanone | 5 | μg/mL furanone | 5 | μg/mL furanone | 5 | μg/mL furanone |
| Well C | 5 | nM OHHL | 10 | nM OHHL | 25 | nM OHHL | 50 | nM OHHL | 100 | nM OHHL |
| | 2.5 | μg/mL furanone | 2.5 | μg/mL furanone | 2.5 | μg/mL furanone | 2.5 | μg/mL furanone | 2.5 | μg/mL furanone |
| Well D | 5 | nM OHHL | 10 | nM OHHL | 25 | nM OHHL | 50 | nM OHHL | 100 | nM OHHL |
| | 1.25 | μg/mL furanone | 1.25 | μg/mL furanone | 1.25 | μg/mL furanone | 1.25 | μg/mL furanone | 1.25 | μg/mL furanone |
| Well E | 5 | nM OHHL | 10 | nM OHHL | 25 | nM OHHL | 50 | nM OHHL | 100 | nM OHHL |
| | 0.63 | μg/mL furanone | 0.63 | μg/mL furanone | 0.63 | μg/mL furanone | 0.63 | μg/mL furanone | 0.63 | μg/mL furanone |
| Well F | 5 | nM OHHL | 10 | nM OHHL | 25 | nM OHHL | 50 | nM OHHL | 100 | nM OHHL |
| | 0.31 | μg/mL furanone | 0.31 | μg/mL furanone | 0.31 | μg/mL furanone | 0.31 | μg/mL furanone | 0.31 | μg/mL furanone |
| Well G | 5 | nM OHHL | 10 | nM OHHL | 25 | nM OHHL | 50 | nM OHHL | 100 | nM OHHL |
| | 0.16 | μg/mL furanone | 0.16 | μg/mL furanone | 0.16 | μg/mL furanone | 0.16 | μg/mL furanone | 0.16 | μg/mL furanone |
| Well H | 5 | nM OHHL | 10 | nM OHHL | 25 | nM OHHL | 50 | nM OHHL | 100 | nM OHHL |
| | 0 | μg/mL furanone | 0 | μg/mL furanone | 0 | μg/mL furanone | 0 | μg/mL furanone | 0 | μg/mL furanone |

The relative activity in each well of the sample plate is found using the standard curve. Three plots are made, one showing relative activity as a function of OHHL concentration, one curve for each furanone concentration. The second plot is relative activity as a function of furanone concentration, one curve for each OHHL concentration. The third is a 3D plot showing relative activity as a function of both OHHL and furanone concentration.

From the second plot, the furanone concentration reducing the relative activity to 40% (ID₆₀) is determined for each OHHL concentration. Finally, a fourth plot is made showing the ID₄₀ values as a function of OHHL concentration.

Results

To enable easy comparison of the strength of the furanones, an inhibition index (IIX₄₀) is calculated for each furanone compound. Each halogenated furanone was tested at 8 different concentrations in the presence of 5 different OHHL concentrations. The inhibitory activity of each compound on the fluorescent phenotype was diminished as the 3-oxo-C6-HSL concentration increased. All 40 fluorescence readings obtained for the compound are presented in FIG. 2A (see Table 1 for structures). For each furanone tested the 40 readings were used to determine the concentration which, at each OHHL concentration, lowered the RFU value to 40% of the untreated sample. The five values obtained, one for each OHHL concentration, were plotted as a function of the OHHL concentration and the gradient of the best straight line passing through the origin was taken as the inhibition index (IIX₄₀). The IIX₄₀ expresses the number of mole of furanone per mmole of OHHL required to inhibit fluorescence to 40%. A low IIX₄₀ value-therefore indicates that a compound is an efficient QSI.

Interestingly, furanone 56 and 30 have the same basic structures as the classic furanones; except it lacks a R₁ side chain and a R₂/R₃ Br atom. The natural compound 2 has an IIX₄₀ 0.75 where as compound 30 and 556 have IIX₄₀ of 0.01 and 0.51 respectively. Compound 2 did not result in bacterial growth inhibition at the concentrations tested (>50 nm) whereas 30 and 56 inhibited growth slightly above 10 and 50 nm respectively).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

| Compound No. | Structure |
|---|---|
| 2(d3) | 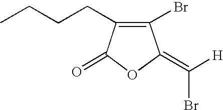 |
| 3(d5) | 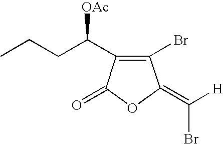 |
| 19 | 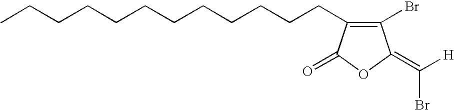 |
| 24 | 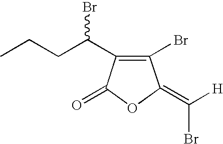 |
| 26 | 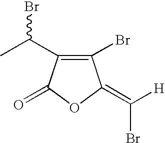 |
| 30 | 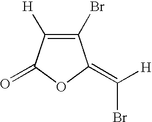 |
| 34 | 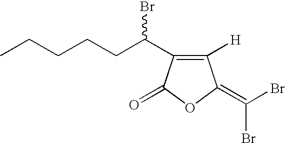 |
| 58 | 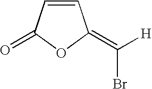 |
| 70 | 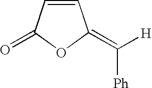 |
| 72 | 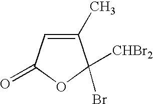 |
| 74 | 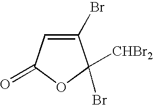 |
| 75 | 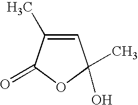 |
| 76 | 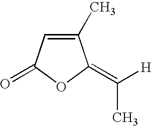 |
| 78 | 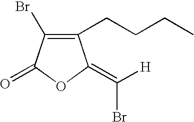 |
| 19 | 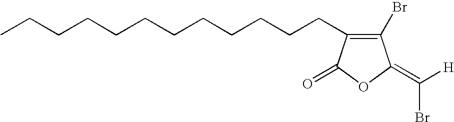 |
| 77 | 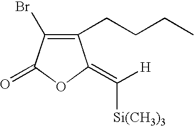 |
| 80 | 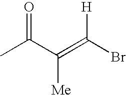 |
| 88 | 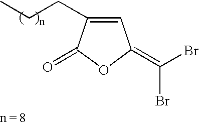 |
| 91 | 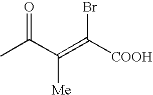 |
| | 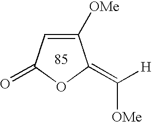 |
| 78 | 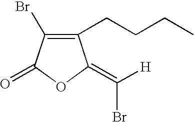 |

TABLE 2

Bacterial strains and plasmids used in this study

| | Relevant genotype and | |
|---|---|---|
| Strains and plasmids | characteristics | Reference |

| | | |
|---|---|---|
| E. coli | | |
| MT102 | F thl araD139 ara- | T. Hansen, Nova |
| | leuΔ7679 Δ(laclOPZY) | Nordisk A/S |
| | galU gal'K r⁻ m⁺ Sm^{R} | |
| CC118 λpir | Δ(ara-leu) araD ΔlacX74 | (Herrero et al., 1990) |
| | galE galK phoA20 thi-1 | |
| | rps-1 rpoB argE(Amp) | |
| | recA thl pro hsRM* | |
| | RP4-2-Tc:Mu-Km::Tn7 | |
| | λpir | |
| P. aeruginosa | | |
| PAO1 | Wild-type P. aeruglnosa | (Holloway, 1955) |
| PAO-JP2 | lasl rhil derivative of | (Pearson et al., 1997) |
| | PAO1, Hg^{R}Tc^{R} | |
| Plasmids | | |
| pJBA25 | Ap^{R}, pUC18NotI RBSII- | (J. B. Andersen, |
| | gfp(ASV)-T₀ − T₁ | unpublished) |
| pJBA132Gm | Tc^{R} Gm^{R}; luxR Pluxl- | (Andersen et al., 2001) |
| | gfp(ASV) | |
| pUCP22Not | Ap^{R} Gm^{R}; | (Herrero et al., 1990) |
| | Pseudomonas-shuttle | |
| | and cloning vector, on | |
| | pRO1614 | |
| pKDT17 | Ap^{R}, lasB-lacZ Plac- | (Pearson et al., 1994) |
| | lasR | |
| pUTTc | Ap^{R} Tc^{R}; Tn5-based | (de Lorenzo et al., 1990) |
| | delivery plasmid | |
| pTn5-Gm | Ap^{R} Gm^{R}; Tn5-based | (Whiteley et al., 2000) |
| | delivery plasmid | |
| pDsRed | Ap^{R}; DsRed expression | CLONTECH |
| | vector | Laboratories, Inc. |
| pTn5-Red | Ap^{R} Tc^{R}; pUTTc | This study |
| | carrying Plac-dsred- | This study |
| | T₀ −T₁ | |
| pMH306 | AP^{R} Gm^{R}; pUCP22Not | This study |
| | carrying P_{A1/04/03}-RBSII- | |
| | gfp(ASV)-T₀ − T₁ | |
| pMH391 | Ap^{R} Gm^{R}; | This study |
| | Pseudomonas-shuttle | |
| | and gfp(ASV)-fusion | |
| | vector with RBSII- | |
| | gfp(ASV)-T₀ − T₁ | |
| pMHLB | Ap^{R} Gm^{R}; pMH391 | This study |
| | carrying PlasB-gfp(ASV) | |
| pMHLAS | Ap^{R} Gm^{R}; pMH391 | This study |
| | carrying PlasB-gfp(ASV) | |
| | Plac-lasR | |
| pTn5-LAS | Ap^{R} Gm^{R}; pTn5-Gm | This study |
| | carrying PlasB-gfp(ASV) | |
| | Plac-lasR | |
| pBAD18 | Ap^{R}; araC P_{BAD} promoter | (Guzman et al., 1995) |
| pBADGfp | Ap^{R} Gm^{R}; | Ths study |
| | Pseudomonas-shuttle | |
| | vector with araC P_{BAD}- | |
| | gfp(ASV) | |
| pTn5-BADGfp | Ap^{R} Gm^{R}; pTn5-Gm | This study |
| | carrying araC P_{BAD}- | |
| | gfp(ASV) | |
| pRK600 | Cm^{r}; on ColE1 RK2- | (Kessler et al., 1992) |
| | Mob* RK2-Tra*; helper | |
| | plasmid in triparental | |
| | conjugations | |
| Primers | lasB fwd | 5′- |
| | | GCTCTAGAGCGGCCA |
| | | GGAAAGCGTGCAA-3′ |
| | lasB rev | 5′- |
| | | GCTGCTGCATGCTTGT |
| | | TCAGTTCTCCTGGT-3′ |
| | laSR fwd | 5′- |
| | | CGGGATCCGGCACGA |
| | | CAGGTTTCCCGAC-3′ |
| | lasR rev | 5′- |
| | | GCCGGCCAGTGCCAA |
| | | GCTTGC-3′ |
| | araCP fwd | 5′- |
| | | GGGTACGTCGACTGA |
| | | TCACCTATGCTACTCC |
| | | GTCAAGCCG-3′ |
| | araCP rev | 5′- |
| | | GCGCTCGTCGACTGA |
| | | TCAGTTCAAATCCGCT |
| | | CCCGGCGG-3′ |

References

Albus, A. M., Pesci E. C., Runyen-Janecky, L. J. Wet. S. E. and Iglewski, B. H. (1997). Vfr controls quorum sensing in Pseudomonas aeruginosa. J Bacterial 179, 3928-3935.

Andersen, J. B., Heydorn. A, Hentzer, M., Eberl, L., Gelsenberger O., Christensen. B. B., Molin, S., and Givskov. M. (2001). gfp based N-acyl-homoserine-lactone monitors for detection of bacterial communicatin. Appl Environ Microbiol 67, 5755.

Andersen, J. B., Sternberg C., Poulsan, L. K., Bjom, S. P., Givskov, M., and Molin, S. (1998) New unstable variants of green fluorescent protein for studies of transient gene expression in bacteria. Appl Environ Microbian 64, 2240-2246.

Anderson, R. M., Zimprich, C. A. and Rust, L. (1999). A Second Operator is Involved in Pseudomonas aeruginosa Elastase (lasB) Activation. J Bacteriol 181, 6246-6270.

Ayora, S. and Gotz, F. (1994). Genetic and biochemical properties of an extracellular neutral metalloprotease from Staphylococcus hyicus subsp. hyicus. Mol Gen Genet 242, 421-430.

Bertani, G. (1961), Studies on lysogensis. I. The mode of phage liberation by lysogenic Escherichia coli. J Bacteriol 62, 293-300.

Bever, R. A. and Iglewski, B. H. (1988). Molecular characterization and nucleotide sequence of the Pseudomonas aeruginosa elastase structural gene. J Bacteriol 170, 4309-4314.

Bloemberg, G. V., O'Toole, G. A., Lugtenberg, B. J. J., and Kolter, R. (1977). Green fluorescent protein as a marker for Pseudomonas spp. Appl Environ Microbriol 63, 4543-4551.

Brint, J. M. and Ohman, D. E. (1995). Synthesis of multiple exoproducts in Pseudomonas aeruginosa is under the control of RHlR-RhiI, another set of regulators in strain PAO1 with homology to the autoinducer-responsive LuxR-LuxI family. J Bacteriol 177, 7155-7163.

Brumlik, M. J. and Storey, D. G. (1992). Zinc and iron regulate translation of the gene encoding Pseudomonas aeruginosa elastase, Mol Microbiol 6, 337-344.

Brumlik, M. J. and Storey, D. G. (1998). Post-transcriptional control of Pseudomonas aeruginosa lasB expression involves the 5′ untranslated region of the mRNA, FEMS Microbiol Lett 159, 233-239.

Chalfie, M., Yu, Y., Eusidrchen, G., Ward. W. W., and Prasher, D. C. (1994). Green fluoroscent protein as a marker for gene expression. Science 263, 802-805.

Charlton, T. S., de Nys, R., Netting, A., Kumar, N., Hentzer, M., Glvskov, M., and Kjelleberg, S. (2000). A novel and sensitive method for the quantification of N-3-oxo-acyl homoserine lactones using gas chromatography-mass spectrometry: application to a model bacterial biofilm. Environmental Microbiology 2, 530-541.

Christensen, B. B., Stemberg, C., Andersen, J. B., Palmer, R. J., Jr., Nielsen, A. T., Glvskov, M., and Molin, S. (1999). Molecular tools for study of biofilm physiology. Methods Enzymol 310, 20-42.

Ciofu, O., Giwercman, B., Pedersen, S. S., and Hoiby, N. (1994) Development of antibiotic resistance in Pseudomonas aeruginosa during two decades of antipseudomonal treatment at the Danish CF Center. APMIS 102, 674-680.

Clark, D. J. and Maalee. O. (1967). DNA replication and the division cycle in Escherichia coli. J Mol Biol 23, 99-112.

Costerton, J. W., Lewandowski, Z., Caldwell, D. E., Korber, D. R., and Lappin-Scott, H. M. (1995). Microbial biofilms. Annu Rev Microbial 49, 711-745.

Costerton, J. W., Stewart, P. S., and Greenberg, E. P. (1999). Bacterial biofilms: a common cause of persistent infections. Science 284, 1318-1322.

Davies, D. G., Parsek, M. R., Pearson, J. P., Iglewski, B. H., Costerton, J. W., and Greenberg, E. P. (1998). The involvement of cell-to-cell signals in the development of a bacterial biofilm. Science 280, 295-298.

de Kievit, T., Seed, P. C., Nexexon, J., Passador, L., and Iglewski, B. H. (1999). Real, a novel repressor of virulence gene expression in Pseudomonas aeruginosa. J Bacteriol 181, 2175-2184.

De Kievit, T. R. and Iglewski, B. H. (2000). Bacterial quorum sensing in pathogenic relationships. Infect Immun. 68, 4839-4849.

de Lorenzo, V., Herrero, M., Jakubzik, U., and Timmis, K. N. (1990). Mini-Tn5 transposon derivatives for insertion mutagenesis, promotor probing, and chromosomal insertion of cloned DNA in gram-negative eubacteria. J Bacteriol 172, 6568-6572.

de Nys, R. Steinberg, P. D., Willemsen, P., Dworjanyn. S. A, Gabelish, C. L., and King. R. J. (1995). Broad spectrum effects of secondary metabolites from the red alga Delisea pulchia in antifouling assays. Biofouling 8, 259-271.

de Nys, R., Wright, A. D., Künig, G. M., and Sticher, O. (1993). New halogenated furanones from the marine alga Delisea pulchra. Tetrahedron 49, 11213-11220.

Olver, J. M., Bryan, L. E., and Sokol, P. A. (1990). Transformation of Pseudomonas aeruginosa by electroporation. Anal Biochem 189, 75-79.

Ebrl, L. (1999). N-acyl homoserinelactone-mediated gene regulation in gram-negative bateria Syst Appl Microbiol 22, 493-506.

Eberl, L., Molin, S., and Givskov, M. (1999). Surface Motility of Serratia liquefaciens MG1 J Bacteriol 181. 1703-1712.

Eberl, L., Winson, M. K. Sternberg, C. Stewart. G. S. A B., Christiansen, G., Chhabra, S. R., Bycoft, B. W., Williams, P., Molin, S., and Givskov, M. (1996). Involvement of N-acyl-L-homoserine lactone autoinducers in controlling the multicellular behviour of Serratia liquefaciens. Mol Microbiol 20, 127-136.

Fukushima, J., Ishiwata, T., You, Z., Ishll, T., Shigematsu, T., Kurata, M., Chikumaru-Fujta, S., Bycroft, B. W. Stewart, G. S., Kawamoto, S., Williams, P., and Okuda, K. (1997). Dissection of the promotor/operator region and evaluation of N-acylhomoserine lactone mediated transcriptional regulation of elastase expression in Pseudomonas aeruginosa. FEMS Microbiol Lett 146, 311-318.

Fuqua, C. and Greenberg, E. P. (1999). Self Percepition in bacteria: quorum sensing with acylated homoserine lactones. Curr Opin Microbiol 1, 183-189.

Fuqua, C., Winans, S. C., and Greenberg, E. P. (1997). Quorum sensing in bacteria: the LuxR-Luxi family of cell density-responsive transcriptional regulators. J Bacteriol 176, 269-275.

Gambello, M. J. and Iglewski, B. H. (1991). Cloning and characterization of the Pseudomonas aeruginosa lasR gene, a transcriptional activator of elastase expression. J Bacteriol 173, 3000-3009.

Gambello, M. J., Kaye, S., and Iglewski, B. H. (1993) LasR of Pseudomonas aeruginosa is a transcriptional activator of the alkaline protease gene (epr) and and enhancer of exotoxin A expression. Infect Immun 61, 1180-1184.

Glvskov, M., de Nys, R., Manefield, M., Gram L., Maximillen, R., Eberl, L., Molin, S., Steinberg, P. D., and Kjelleberg, S. (1996). Eukaryotic interference with homoserine lactone-mediated prokayotic signalling. J Bacteriol 178, 6618-6622.

Givskov, M., Eberl, L., and Molin, S., (1997). Control of axoenzyme production, motility and cell differentiation in Serralia liquefaciens. FEMS Microbiol Lett 148, 115-122.

Glvskov, M., Ostting, J., Eberl, L., Lindum, P. W., Christensen, A. B., Christiansen, G., Molin, S., and Kjelleberg, S. (1998) Two separate regulatory systems participate in control of swarming motility of Serratia liquefaciens MG1. J Bacteriol 180, 742-745.

Glassner, A., Smith, R. S., Iglewski, B. H., and Robinson, J. B. (1999). Roles of Pseudomonas aeruginosa las and rhl Quorum-Sensing Systems in Control of Twitching Motility. J Bacteriol 181, 1623-1629.

Gray, K. M., Passador, L., Iglewski, B. H., and Greenberg, E. P. (1994). Interchangeability and specificity of components from the quorum-sensing regulartory systems of Vibrio fischeri and Pseudomonas aeruginosa. J Bacteriol 176, 3076-3080.

Greenberg, E. P. (1997). Quorum sensing in gram-negative bacteria. ASM News 63, 371-377.

Guzman, L. M., Belin, D., Carson, M. J., and Beckwith, J. (1996). Tight regulation, modulation, and high-level expression by vectors containing the arabinosa P_{BAD} promotor. J Bacteriol 177, 4121-4130.

Hassett, D. J., Ma, J. F., Elkins, J. G., McDermott, T. R., Ochsner, U. A., West, S. E., Huang, C. T., Fredericks, J., Burnett, S., Stweart, P. S., McFeters, G., Passador, L., and Iglewski, B. H. (1999). Quorum sensing in Pseudomonas aeruginosa controls expression of catalase and superoxide dismutase genes and mediates biofilm suspectibility to hydrogen peroxide. Mol Microbiol 34, 1082-1093.

Heeb. S., Itoh, Y., Nishijyo, T., Schnider, U., Keel, C., Wada J., Walsh U., O'Gara, F., and Haas D. (2000). Small, stable shuttle vectors based on the minimal pVS1 replicon for use in gram-negative, plant associated bacteria. Mol Plant Microbe Interact 13, 232-237.

Herrero, M., de Lorenzo, V., and Timmis, K. N. (1990). Transposon vectors containing non-antibiotic resistance selection markers for cloning and stable chromosomal insertion of foreign genes in gram-negative bacteria. J Bacteriol 172, 6557-6567.

Hoiby, N. (1977). Pseudomonas aeruginosa infection in cystic fibrosis. Diagnostic and prognostic significance of Pseudomonas aeruginosa precipitins determined by means of crossed immunoelectrophoresis. A survey, Acta Pathol Microbiol Scand Suppl 1-95.

Hoiby, N. (2000). Prospects for the prevention and control of pseudomonal infection in children with cystic fibrosis. Paediatr Drugs 2, 451-483.

Hoiby, N. and Koch, C. (1990). Pseudomonas aeruginosa infection in cystic fibrosis and its management. Thorax 45, 881-884.

Holloway, B. W. (1955). Genetic Recombination in Pseudomonas aeruginosa. J Gen Microbiol 13, 572-581.

Kessler, B., de, Lorenzo, V., and Timmis, K. N. (1992). A general system to integrate lacZ fusions into the chromosomes of gram-negative eubacteria: regulation of the Prn promoter of the TOL plasmid studied with all controlling elements in monocopy. Mol Gen Genet 233, 293-301.

Kjelleberg, S., Steinberg, P. D., Givskov, M., Gram, L., Manefield, M., and de Nys R. (1997). Do marine natural products interfere with prokaryotic AHL regulatory systems?Aquat Microb Ecol 13, 85-93.

Kleerebezem, M., Quadri, L. E., Kuipers, O. P., and de Vos, V. M. (1997). Quorum sensing by peptide pheromones and two-component signal-transduction systems in Gram-positive bacteria. Mol Microbiol 24, 895-904.

Kline, T., Bowman, J., Iglewski, B. H., de Kievit, T., Kakai, Y., and Passador, L., (1999). Novel synthetic analogs of the Pseudomonas autoinducer. Bioorg Med Chem Lett 9, 3447-3452.

Koch, C. and Hoiby, N. (1993). Pathogensis of cystic fibrosis. Lancet 341, 1065-1069.

Lam, J., Chan R., Lam, K., and Costerton, J. W. (1980). Production of mucoid microcolonies by Pseudomonas aeruginosa within infected lungs in cystic fibrosis. Infect Immun. 28, 546-556.

Latiffl, A., Foglino, M., Tanaka, K., Williams, P., and Lazdunski, A. (1996). A hierarchical quorum-sensing cascade in Pseudomonas aeruginosa links the transcriptional activators LasR and RhlR (VsmR) to expression of the stationary-phase sigma factor RpoS. Mol Microbiol 21, 1137-1145.

Lindum, P. W., Anthonl, U., Christophersen, C., Eberl, L., Molin, S., and Givskov, M. (1998). N-Acyl-L-homoserine lactone autoinducers control production of an extracellular lipopeptide biosurfactant required for swarming motility of serratia liquefaciens MG1. J Bacteriol 180, 6384-6388.

Manefiled, M., de Nys, R., Kumar, N., Read, R., Givskov, M., Steinberg, P. D., and Kjelleberg, S. (1999). Evidence that halogenated furanones from Dellsea pulchra inhibit acylated homoserine lactone (AHL)-mediated gene expression by displacing the AHL signal from its receptor protein. Microbiology 145, 283-291.

Manefield, M., Harris, L., Rice, S. A., de Nys, R., and Kjelleberg, S. (2000). Inhibition of luminscence and virulence in the black tiger prawn (Penaeus monodon) pathogen Vibria harveyi by intercellular signal antagonists. Appl Environ Microbial 56, 2079-2084.

O'Toole, G. A. and Kolter, R. (1998). Flagellar and twitching motility are necessary for Pseudomonas aeruginosa biofilm development. Mol Microbiol 30, 295-304.

Ochsner, U. A. and Reiser, J. (1995). Autoinducer-mediated regulation of rhamnolipid biosurfactant synthesis in Pseudomonas aeruginosa. Proc Natl Acad Sci U S A 92, 6424-6428.

Ohman, D. E., Cryz, S. J. Jr, and Iglewski, B. H. (1980). Isoloation and characterization of Pseudomonas aeruginosa PAO mutant that produces altered elastase. J Bacteriol 142, 836-842.

Parsek, M. R. and Greenberg, E. P. (2000). Acyl-homoserine lactone quorum sensing in gram-negative bacteria: a signalling mechanism involved in associations with higher organisms. Proc Natl Acad Sci U S A 97, 8789-8793.

Passador, L., Cook, J. M., Gambello, M. J., Rust, L., and Iglewski, B. H. (1993). Expression of Pseudomonas aeruginosa virulence genes requires cell-to-cell communication. Science 260, 1127-1130.

Passador, L. and Iglewski, B. H. (1993). Quorum sensing and virulence gene regulation in Pseudomonas aeruginosa. In Virulence Mechanisms of Bacterial Pathogens, pp. 65-78. Edited by J. A. Roth. Washington, D.C.: American Society for Microbiology.

Passador, L., Tucker, K. D., Guetin, K. R., Journet, M. P., Kends, A. S., and Iglewski, B. H. (1996). Fuctional analysis of the Pseudomonas aeruginosa autoinducer PAI. J Bacteriol 178, 5885-6000.

Pearson, J. P., Gray, K. M., Passador, L., Tucker, K. D., Eberhard, A., Iglewski, B. H., and Greenberg, E. P. (1994). Structure of the autoinducer required for expression of Pseudomonas aeuroginosa virulence genes. Proc. Natl Acad Sci U S A 91, 197-201.

Pearson, J. P., Passador, L., Iglewski, B. H., and Greenberg, E. P. (1995). A second N-acylhomoserine lactone signal produced by Pseudomonas aeruginosa. Proc Natl Acad Sci U S A 92, 1490-1494.

Pearson, J. P., Pesci, E. C., and Iglewski, B. H. (1997). Roles of Pseudomonas aeruginosa las and rhl quorum-sensing systems in control of elastase and rhamnolipid biosynthesis genes. J Bacteriol 179, 5756-5767.

Pedersen, S. S., Hoiby, N., Espersen, F., and Kock, C. (1992). Role of alginate in infection with mucoid Pseudomonas aeruginosa in cystic fibrosis. Thorax 47, 6-13.

Pesci, E. C. and Iglewski, B. H. (1997). The chain of command in Pseudomonas quorum sensing. Trends Microbiol 5, 132-134.

Pesci, E. C., Millbank, J. B., Pearson, J. P., McKnight, S., Kende, A. S., Greenberg, E. P., and Iglewski, B. H. (1999). Quinolone signalling in the cell-to-cell communication system of Pseudomonas aeruginosa. Proc Natl Acad Sci U S A 96, 11229-11234.

Piper, K, R., von Bodman, S. B., and Farrand, S. K., (1993). Conjugation factor of Agrobacterium tumefaciens regulates Ti plasmid transfer by autoinduction. Nature 362, 448-450.

Pollack, M. (1990) Pseudomonas aeruginosa. In Principles and Practice of Infectious Diseases, pp. 1673-1691. Edited by G. L. Mandell, R. G. Douglas, and J. E. Bennett. New York, N.Y.: Churchill Livingstone Inc.

Rasmussen, T. B., Manefield, M., Andersen, J. B., Eberl, L., Anthoni, U., Christophersen, C., Steinberg, P., Kjelleberg, S., and Givskov, M. (2000). How Delisea pulchra furanones affect quorum sensinig and swarming motility in Serratia liquefaciens MG1. Microbiology 146, 3237-3244.

Reichelt, J. L. and Borowitzka, M. A. (1984). Antimicrobial activity from marine algae: results of a large-scale screening programme. Hydrobiology 116/117. 158-168.

Reimmann, C., Beyelar, M., Latifi, A., Winteler, H., Foglino, M., Lazdunski, A., and Haas D. (1997). The global activator GacA of Pseudomonas aeruginosa PAO positively controls the production of the autoinducer N-butyryl-homoserine lactone and the formation of he virulence factors pyocyanio cyanide, and lipase. Mol Microbiol 24, 309-319.

Rust, L., Pesci, E. C., and Iglewski, B. H., (1996). Analysis of the Pseudomonas aeruginosa elatase (lasB) regulatory region. J Bacteriol 178, 1134-1140.

Sambrook, J., Fritsch, E. F., and Maniatis, T. (1989). Molecular cloning: A laboratory manual 1-3, 2 ed. edn. Cold Spring Harbor, N.Y.: Cold Spring Harbor Laboratory Press.

Schlictman, D., Kubo, M., Shankar, S., and Chakrabarty, A. M. (1985). Regulation of nucleoside diphosphate kinase and secretable virulence factors in Pseudomonas aeruginosa: roles of algR2 and algH. J Bacteriol 177, 2469-2474.

Schweizer, H. P. (1991). Improved broad-host-range lac-based plasmid vectors for the isolation and characterization of protein fusions in Pseudomonas aeruginosa. Gene 103, 87-92.

Seed, P. C., Passador, L., and Iglewski, B. H. (1995). Activation of the Pseudomonas aeruginosa lasI gene by LasR and the Pseudomonas autoinducer PAI: an autoinduction regulatory hierarchy. J Bacteriol 177, 554-659.

Singh, P. K., Schaefer, A. L., Parsek, M. R., Moninger, T. O., Welsh, M. J., and Greenberg, E. P. (Oct. 12, 2000). Quorum-sensing signals indicate that cytistic fibrosis lungs are infected with bacterial biofilms. Nature 407, 762-764.

Swift, S., Throup, J. P., Williams, P., Salmond, G. P., and Stewart, G. S. (1998). Quorum sensing: a population-density component in the determination of bacterial phenotype. Trends Biochem Sci 21, 214-219.

Todar, D. S., Ferrell, S. J., Nezezon, J. L., Rust, L., and Iglewski, B. H. (1994) lasA and lasB genes of Pseudomonas aeruginosa: analysis of transcription and gene product activity. Infect Immun 62, 1320-1327.

Toder, D. S., Gambello, M. J., and Iglewski, B. H. (1991). Pseudomonas aeruginosa LasA: a second elastase under the transcriptional control of lasR. Mol Microbiol 5, 2003-2010.

Van Delden, C. and Iglewski, B. H. (1998). Cell-to-cell signalling and Pseudomonas aeruginosa infections. Emerg Infect Dis 4, 551-560.

von Bodman, S. B. and Farrand, S. K. (1995). Capsular polysaccharide biosynthesis and pathogenicity in Erwinia stewartii require induction by an N-acylhomoserine lactone autoinducer. J Bacteriol 177, 5000-5008.

West, S. E., Schweizer, H. P., Dall, C., Sample, A. K., and Runyenjanecky, L. J. (1994). Construction of improved Escherichia-Pseudomonas shuttle vectors derived from pUC18/19 and sequence of the region required for their replication in Pseudomonas aeruginosa. Gene 148, 81-86.

Whiteley, M., Lee, K. M., and Greenberg, E. P. (1999). Identification of genes controlled by quorum sensing in Pseudomonas aeruginosa. Proc Natl Acad Sci U S A 95, 13904-13909.

Whiteley, M., Parsek, M. R., and Greenberg, E. P. (2000). Regulation of quorum sensing by RpoS in Pseudomonas aeruginosa. J Bacteriol 182, 4356-4360.

Williams, P., Camara M., Hardman, A., Swift, S., Milton, D., Hopa, V. J., Winzer, K., Middleton, B., Pritchard, D. L., and Bycroft, B. W. (2000) Quorum sensing and the population-dependent control of virulence. Philos Trans R Soc Lond B Biol Sci 355, 667-680.

Winson, M. K., Camara, M., Latifi, A., Foglino, M., Chharbra, S. R., Daykin, M., Bally, M., Chapon, V., Salmond, G. P., Bycroft, B. W., Lazdunski, A., Stewart, G. S. A. B., and Williams, P. (1995). Multiple N-acyl-L-homoserine lactone signal molecules regulate production of virulence determinants and secondary metabolites in Pseudomonas aeruginosa. Proc Natl Acad Sci U S A 92, 9427-9431.

Wu, H., Song, Z., Hentzer, M., Andersen, J. B., Hedom, A., Mathee, K., Moser, C., Eberl, L., Molin, S., Hoiby, N., and Givskov, M. (2000). Detection of N-acylhomoserine lactones in lung tissues of mice infected with Pseudomonas aeruginosa. Microbiology 146, 2481-2493.

1. A methed of degrading or causing sloughing of a biofilm the method comprising applying to the biofilm at least one compound of general formula I: 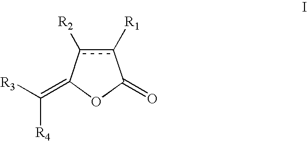
wherein R₄ is selected from H, halogen, alkyl, alkoxy, acyl, alkenyl, aryl, alkylaryl or arylalkyl whether unsubstituted or substituted, optionally interrupted by one or more heteroatoms, straight chain or branched chain, hydrophilic or fluorophilic;
R₂, R₃ and R₄, which may be the same or different, are independency selected from H, halogen, alkyl, alkoxy, acyl, alkenyl, aryl, alkylaryl, arylalkyl, or a silyl group, whether unsubstituted or substituted, optionally interrupted by one or more heteroatoms, straight chain or branched chain, hydrophilic or fluorophilic;
R₃ or R₄+R₂ can be a saturated or an unsaturated cycoalkane; and
“═” represents a single bond or a double bond,
or a compound of general formula II 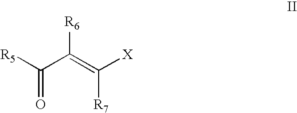
wherein R₅, R₆ and R₇, which may be the same or different, are independently selected from H, halogen, alkyl, alkoxy, alkenyl, alklynl, aryl, arylalkyl, carboxyl, acyl, acyloxy, acylamino, formyl and cyano whether unsubstituted or substituted, optionally interrupted by one or more hetero atoms, straight chain or branched chain, hydrophilic, hydrophobic or fluorophilic and X is a halogen.

2. A method of degrading or causing sloughing of a Pseudomonas biofilm in the lung of a subject suffering from cystic fibrosis, the method comprising administering to the biofilm at least one compound of general formula I: 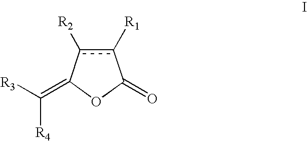
wherein R₁ is selected from H, halogen, alkyl, alkoxy, acyl, alkenyl, aryl, alkylaryl or arylalkyl whether unsubstituted or substituted, optionally interrupted by one or more heteroatoms, straight chain or branched chain, hydrophilic or fluorophilic;
R₂, R₃ and R₄, which may be the same or different, are independently selected from H, halogen, alkyl, alkoxy, acyl, alkenyl, aryl, alkylaryl, arylalkyl, or a silyl group, whether unsubstituted or substituted, optionally interrupted by one or more heteroatoms, straight chain or branched chain, hydrophilic or fluorophilic;
R₃ or R₄+R₂ can be a saturated or an unsaturated cycloalkane; and
“═” represents a single bond or a double bond.
or a compound of general formula II 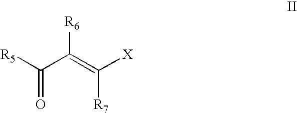
wherein R₆ and R₇ are independently H, halogen, carboxyl, ester, formyl, cyano, alkyl, alkoxy, axoalkyl, alkenyl, aryl or arylalkyl whether unsubstituted or substituted, optionally interrupted by one or more heteroatoms, straight chain or branched chain, hydrophilic or fluorophilic;
X is a halogen;
R₅ is H, alkyl, alkenyl, alkynyl, alkene, alkyne, aryl, arylalkyl, whether unsubstituted or substituted, optionally interrupted by one or more heteroatoms, straight chain or branched chain, hydrophilc or fluorophilic.

3. A method according to claim 1 for treating a disease or infection in a human or animal subject in which a biofilm is formed, the method comprising administration to the subject of a therapeutically or prophylatically effective amount of the composition.

4. A method according to claim 3, wherein the disease or infection is selected from periodontial disease, tooth decay, prosate infections, kidney stones, tuberculosis, Legionnaire's disease, an infection of the middle ear, burn and/or wound infection.

5. A method according to any one of claims 1 to 5, wherein the method involves treating an immuno-compromised individual.

6. A biofilm degrading or sloughing composition comprising an amount of a compound comprising at least one compound of general formula I: 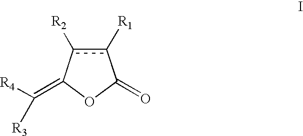
wherein R₁ Is selected from H, halogen, alkyl, alkoxy, acyl, alkenyl, aryl, alkylaryl or arylalkyl whether unsubstituted or substituted, optionally interrupted by one or more heteroatoms, straight chain or branched chain, hydrophilic or fluorophilic;
R₂, R₃ and R₄, which may be the same or different, are independently selected from H, halogen, alkyl, alkoxy, acyl, alkenyl, aryl, alkylaryl, arylalkyl, or a silyl group, whether unsubstituted or substituted, optionally interrupted by one or more heteroatoms, straight chain or branched chain, hydrophilic or fluorophilic;
R₃ or R₄+R₂ can be a saturated or an unsaturated cycloalkane; and
“═” represents a single bond or a double bond,
or a compound of general formula II 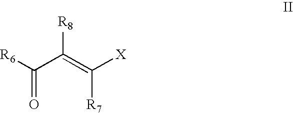
wherein R₅, R₆ and R₇, which may be the same or different, are independently selected from H, halogen, alkyl, alkoxy, alkenyl, alkynyl, aryl aryalkyl, carboxyl, acyl, acyloxy, acylamino, formyl and cyano whether unsubstituted or substituted, optionally interrupted by one or more hetero atoms, straight chain or branched chain, hydrophilic, hydrophobic or fluorophilic and X is a halogen.
wherein the amount of the compound(s) in the composition is effective to degrade or causes sloughing of at least a part of the biofilm.

7. A method or composition of any one of the preceding claims, wherein the compound comprises at least compound of the formula:

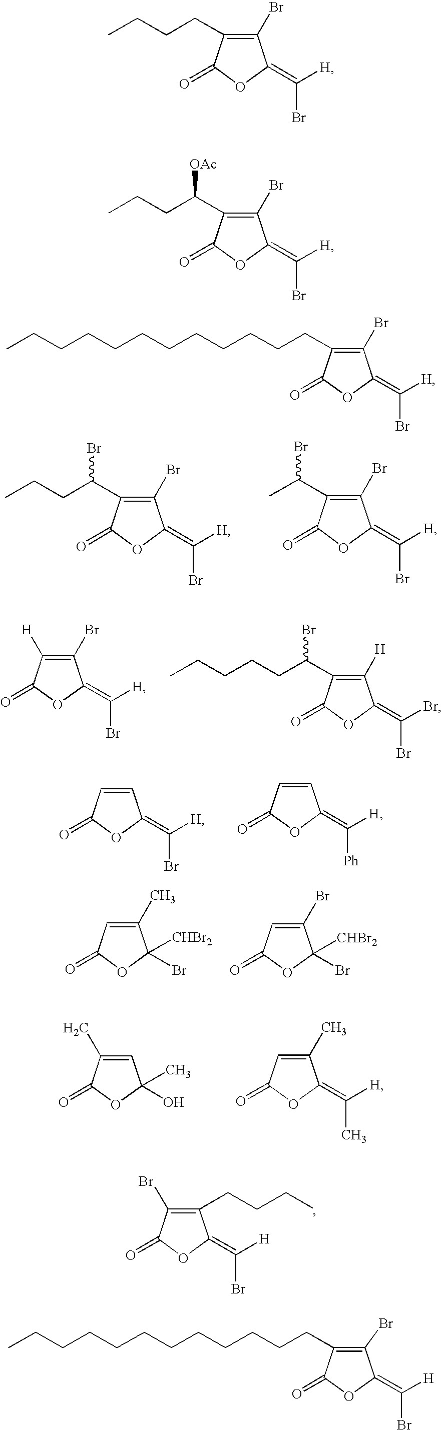

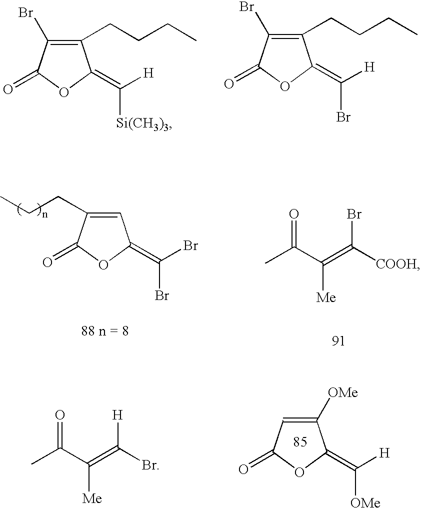

8. The composition of claim 6 or 7, additionally comprising a surfactant selected from the group consisting of anionic, nonionic, amphoteric, biological surfactants and mixtures thereof.

9. The composition of claim 8, wherein the surfactant is sodium dodecyl sulfate.

10. A composition of any one of claims 6 to 9, further comprising a compound selected from the group consisting of biocides, fungicides, antibiotics, and mixtures thereof.

11. A method of removing biofilm at least in part from a surface comprising the step of applying a cleaning-effective amount of the composition of according to any one of claim 6 to 10 to a biofilm containing surface.

12. A method of claim 1, wherein the surface is a hard, rigid surface.

13. A method of claim 12, wherein the surface is selected from the group is consisting of a drainpipe, glaze, ceramic, porcelain, glass, metal, wood, chrome, plastic, vinyl, formica, flooring, and operating theatre surfaces.

14. A method of claim 11, wherein the surface is a soft, flexible surface.

15. A method of claim 14, wherein the surface is selected from the group consisting of shower curtains or liners, upholstery, laundry and carpeting.

16. A method or composition according to any one of the preceding claims, wherein the biofilm comprises a bacteria selected from the class Pseudomonas, Staphylococcus Aeromonas, Burkholderia, Erwinia Fusobacterium, Helicobacter, Klebslella, Listeria, Mycobacterium, Neisseria, Porphymomonas, Providencia, Ralstonia, Salmonella, Streptoccus, Vibrio, Xenorhabus, and Yersinia and combinations of two or more thereof.

17. A method according to claim 16, wherein the bacteria is selected from at least one of the group consisting of Aeromonas hydrophilia, A. salmonicida, Burkholderia cepacia, Enterobacter aerogenes, Escherichia coli, Erwinia carotovora, Fusobacterium nucleatum, Helicobacter pylori, Kiebsiella pneumonia, Listerla monocytogenes, Myrobacterium tuberculosis, Nelsseria meningitis, N. gonorrhea, Porphyomonas gingivitis, Providencia stuartii, Pseudomonas aeruginosa, Ralstonia solanacearum, Salmonella typhimurium, Salmonella cholerasuis, Serratia liquefaciens, S. marcesens, Staphylococcus aureus, S. epidermidis, Streptococcus mutans (sobrinus), Strep, pyogenes, Strep pneumonia, Vibrio parahaemolyticus, V. vulnificus, V. cholerae, V. harveyl, V. angullarum, Xenorharbus nemotophilius, Yersinia pastis, Y. enterocolitica, and Y. pseudotuberculosis.

18. A dentifrice comprising an effective amount of a composition pf claim 6.

19. A mouthwash comprising an effective amount of a composition of claim 6.

20. A method for treatment of dental caries comprising administering an effective amount of a composition of claim 6.

21. A method of prevention of dental caries comprising administering an effective amount of a composition of claim 6.

22. A method of treatment of acne comprising topically administering an effective amount of a composition of claim 6.

23. A composition according to claim 8, useful for flushing a catheter and having activity against microorganisms in established biofilms.

24. A composition according to claim 23 further comprising a biocide and/or an antibiotic.

25. A method of treating a medical indwelling device having a biofilm formed on at least a part of a surface thereof, the method comprising contacting the device with a composition in accordance with claim 6.

26. A method according to claim 25, wherein the indwelling device is selected from the group consisting of bone prostheses, surgical pins, heart valves, pacemakers and the like.

27. A method for inhibiting microbially influenced corrosion of microbially influenced corrosion-susceptible metal surfaces having an anerobic biofilm containing active sulfate-reducing bacteria comprising containing the biofilm with a composition according to claim 6.

28. A method of removing biofilm from the surfaces of conduit comprising contacting at least part of the circuit surface with a composition according to claim 6.

29. A method according to claim 28, wherein the method further comprises means to induce turbulence to assist in removal of the biofilm.

30. A method according to claim 28 or 29, wherein the composition is introduced into the conduit with one or more surfactants.

31. A method according to any one of claims 28 to 30, wherein the conduits are piping conduits in industrial facilities or in household plumbing systems.

32. A method of treating a biofilm in a cooling water system comprising contacting at least part of a cooling water in the system with a composition in accordance with claim 6.

33. A method according to claim 32, wherein the cooling system is that used in power-generating plants, refineries, chemical plants, or air conditioning systems and the like.

34. An ophthalmic composition for treating biofilm formation comprising an effective amount of a composition according to claim 8, and a bactericidal agent to kill individual bacteria that are released from the biofilm structure as it is being degraded or sloughed.

35. A composition as claimed in claim 34 wherein the bactericidal agent is selected from the group consisting of: aminoglycoside antibiotic; a quinolone or fluoroquinolone antibiotic; a cephaloosporin antibiotic; a penicllin antibiotic; and tobramycin.

36. A composition as claimed in claim 35, wherein the bactericidal agent is selected from the group consisting at ciprofloxacin, ofloxacin, aztreonam, vancomycin, streptomycin, neomycin, and gentamicin.

37. An ophtlamic composition according to claim 35, which is suitable for treating an infection of the eye.

38. A method of cleaning and disinfecting a contact lens comprising contacting the lens with an effective amount of a composition of claim 6.

39. A method according to claim 38, wherein the composition is in the form of a saline solution.

40. A method according to claim 11, wherein the surface is a living surface membrane or skin.

41. A method according to claim 40, wherein the surface is a tissue, membrane or skin.
